(19) United States
(12) Patent Application Publication
(10) Pub. No.: US 2025/0296955 A1
(43) Pub. Date: Sep. 25, 2025

(54) PIN1 TARGETING COMPOUNDS AND DEGRADERS AND METHODS THEREOF

(71) Applicant: The Regents of the University of California, Oakland, CA (US)

(72) Inventors: Maurizio Pellecchia, San Diego, CA (US); Giulia Alboreggia, Moreno Valley, CA (US); Parima Udompholkul, Riverside, CA (US)

(21) Appl. No.: 19/087,077

(22) Filed: Mar. 21, 2025

Related U.S. Application Data
(60) Provisional application No. 63/568,786, filed on Mar. 22, 2024.

(51) Int. Cl. C07K 5/08; A61K 38/00

(52) U.S. Cl. CPC C07K 5/0802; A61K 38/00

(57) ABSTRACT

Certain embodiments of the invention provide new compounds, conjugates, and salts as described herein that may inhibit and/or degrade PIN1. Also described methods of inhibiting and/or degrading PIN1 and methods of treating a PIN1 associated disease, and methods of developing degrader compounds for a target protein.

### CROSS-REFERENCE TO RELATED APPLICATION

This application claims priority to U.S. Provisional Application No. 63/568,786 that was filed on Mar. 22, 2024. The entire content of the application referenced above is hereby incorporated by reference herein.

### GOVERNMENT FUNDING

This invention was made with government support under CA242620 and CA285114 awarded by the National Institutes of Health. The government has certain rights in the invention.

### BACKGROUND OF THE INVENTION

Proline-directed phosphorylation is a signaling event that is central to the activation of several cancer mechanisms, regulating both oncoproteins and tumor suppressors. These signaling events are further regulated by the proline isomerase Pin1 that recognizes specific pSer-Pro or pThr-Pro motifs. Pin1 overexpression is a major contributor to tumorigenesis, potentially activating oncoproteins and/or inactivating tumor suppressors. Past attempts to identify Pin1 inhibitors have met certain challenges, resulting in agents with various limitations or unsatisfactory properties. New compounds that effectively modulate this target are needed.

### SUMMARY OF THE INVENTION

Certain embodiments provide a compound described herein (e.g., in Example 1).

Certain embodiments provide a compound or conjugate of Formula (I)

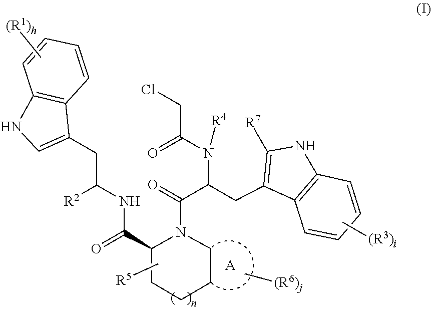

or a salt thereof, wherein
each R¹ is independently halo, hydroxy, (C₁-C₆)alkyl, (C₁-C₆)alkoxy, —CN, —NO₂, —SO₂F, —OSO₂F, or (C₁-C₆)alkyl-(C₁-C₆)alkoxy;
R² is H, (C₁-C₆)alkyl, aryl, heteroaryl, heterocycle, —C(═O)-L-D, —C(═O)NH₂, or —C(═O)NH—Rₐ, wherein the (C₁-C₆)alkyl, aryl, heteroaryl, or heterocycle is optionally substituted with one or more substituents selected from the group consisting of halo, hydroxy, (C₁-C₆)alkyl, (C₁-C₆)alkoxy, —CN, —NO₂, —SO₂F, and —OSO₂F;
each R³ is independently halo, hydroxy, (C₁-C₆)alkyl, (C₁-C₆)alkoxy, —CN, —NO₂, —SO₂F, —OSO₂F, or (C₁-C₆)alkyl-(C₁-C₆)alkoxy;
h, i, and j are each independently 0, 1, 2, 3, or 4;
R⁴ is H, (C₁-C₆)alkyl, (C₂-C₆)alkenyl, or (C₂-C₆)alkynyl and R⁷ is H; or R⁴ and R⁷ taken together are —CH₂—;
R⁵ is absent, halo, (C₁-C₆)alkyl, (C₁-C₆)alkoxy, —CN, —NO₂, —SO₂F, —OSO₂F, (C₁-C₆)alkyl-(C₁-C₆)alkoxy, aryl, heteroaryl, or heterocycle;
n is 0 or 1;
ring A is absent or a (C₃-C₆) carbocycle ring;
each R⁶ is independently halo, hydroxy, (C₁-C₆)alkyl, (C₁-C₆)alkoxy, —CN, —NO₂, —SO₂F, or —OSO₂F;
Rₐ is (C₁-C₆)alkyl, aryl, heteroaryl, or heterocycle, wherein the (C₁-C₆)alkyl, aryl, heteroaryl, or heterocycle is optionally substituted with one or more substituents selected from the group consisting of halo, hydroxy, (C₁-C₆)alkyl, (C₁-C₆)alkoxy, —CN, —NO₂, —SO₂F, and —OSO₂F;
L is absent or is a linking group; and
D is the residue of an E3 ligase targeting drug moiety, the residue of another molecule targeting PIN1, or the residue of a ligand targeting a different protein target to induce its PIN1 driven degradation.

Certain embodiments provide a method of inhibiting and/or degrading PIN1 in vitro or in vivo, comprising contacting PIN1 with a compound, conjugate, or salt described herein.

Certain embodiments provide a method of treating a PIN1 associated disease in a mammal in need thereof, comprising administering a therapeutically effective amount of a compound, conjugate, or salt described herein, to the mammal.

Certain embodiments provide a method of developing degrader compound for a target protein, comprising
contacting a test compound with the target protein in vitro,
determining the binding affinity of the test compound for the target protein,
determining the thermal stability of the target protein in the absence and presence of the test compound,
identifying the test compound as a degrader compound wherein the test compound is determined to be capable of binding the target protein and is determined to be capable of reducing the thermal stability of the target protein.

### BRIEF DESCRIPTION OF THE FIGURES

FIGS. 1A-1B. Expression of Pin1 in cells and comparison of Pin1 degradation in response to Pin1 inhibitors in BxPC3 cells. FIG. 1A) Different cell lines were probed for endogenous expression of Pin1. FIG. 1B) BxPC3 cells were treated with 0.1% DMSO or 25 μM of known Pin1 inhibitors for 24 h and Pin1 levels were quantified.

FIGS. 2A-2D. Comparative properties of selected Pin1 binding agents. FIG. 2A) Chemical structures of D-peptide, BJP-07-017-3, and 158D11. FIG. 2B) Associated DELFIA displacement dose response curves (Standard deviation values are obtained from duplicate measurements) and FIG. 2C) Denaturation thermal shift curves relative to the indicated agents. Curves are normalized in Y axis for better illustration; original data are reported as FIG. 11.

FIG. 2D) Surface representation of Pin1 in complex with BJP-07-017-3 (PDB ID 6034). Residue Cys 113 is highlighted.

FIGS. 3A-3B. Effect of selected agents to Pin1 degradation in cell. Agents selected based on DELFIA and ΔTₘ values were tested for Pin1 degradation in the pancreatic cancer cell line BxPC3. BxPC3 treated with 5 μM (FIG. 3A) or 15 μM (FIG. 3B) of different compounds for 24 h.

FIGS. 4A-4F. Effect of 158H9 and 164A10 on Pin1 degradation in a panel of human cancer cell lines. Agents selected based on DELFIA and ΔTₘ values were tested for Pin1 degradation in FIG. 4A) BxPC3, FIG. 4B) MIA PaCa-2, FIG. 4C) PANC-1, FIG. 4D) MDA-MB-231, FIG. 4E) PC3 and FIG. 4F) A549 NucLight Red. Cells were treated with increasing concentrations of each compound for 24 h.

FIGS. 5A-5B. Pin1 targeting agents cause Pin1 degradation via the ubiquitin-proteasome pathway. BxPC3 cells were treated with FIG. 5A) 158H9 or FIG. 5B) 164A10 in the presence or absence of autophagy (Baf A1 and HCQ) or proteosome (BTZ or CFZ) inhibitors for 24 h.

FIGS. 6A-6C. Characterization of ligand binding to Pin1 by solution NMR spectroscopy. FIG. 6A) Overlay of 2D [¹⁵N,¹H] correlation spectra for Pin1 (50 μM in buffer 50 mM phosphate pH=7.5, 150 mM NaCl and 1 mM DTT) collected in absence (blue) and presence (red) of compound 164A9 (250 μM). Resonance assignments are reported as FIG. 13. FIG. 6B) Ribbon representation of Pin1 in complex with BJP-07-017-3 (stick model) PDB ID 6O34). The secondary structure elements are labeled, and the dark gray areas of the protein represent backbone residues that experienced significant chemical shift perturbations upon complex formation with compound 164A9. FIG. 6C) Overlay of 2D [15N, 1H] correlation spectra for Pin1 (50 μM in buffer 50 mM phosphate pH=7.5, 150 mM NaCl and 1 mM DTT) collected in absence (blue) and presence of various compounds, each at 250 μM: red, compound sulfopin; yellow, compound 164A9; green, compound 158F10. The insert highlights the chemical shift perturbations induced by the ligands on the side chain ¹⁵N^{ε}, ¹H^{ε} indole resonances of residue Trp 73.

FIGS. 7A-7D. Overall structure of hPin1 R14A and the feature-enhanced 2mFₒ-DF_{c} electron density of Cys113 and the covalently bound 158A10, 158D9, and 158F10 compounds. FIG. 7A) The overall structure of hPin1 R14A with bound PEG400 within the asymmetric unit of the P3₁21 crystal form. The PPIase and WW domains of hPin1 are shown. The dashed line outlines the regions featured in the remaining panels. FIGS. 7B-7D) Feature-enhanced 2mFₒ-DF_{c} electron density of Cys113 and the covalently bound 164A10 (PDB ID 8VJG), 158D9 (PDB ID 8VJF), and 158F10 (PDB ID 8VJE) compounds contoured at 1σ, represent as mesh. The inhibitors are shown in ball- and -stick representation.

FIGS. 8A-8C. The 158F10-induced structural changes of Pin1. Structure of Pin1 with bound 158F10 as seen in the single copy (FIG. 8A) and the two copies (FIG. 8B and FIG. 8C) of the inhibitor-bound protein of the P3121 crystal and the P212121 crystal, respectively. Note the conformational flexibility of 158F10 and of Lys 117. See also (PDB ID 8VJE and PDB ID 8VID for the 158F10 protomer).

FIG. 9. Schematic illustration of the proposed mechanism of how a molecular crowbar induces Pin1 degradation. Top panels, binding of agents to Pin1 brings the reactive acetyl group of the inhibitor close to Cys113. Upon formation of a covalent bond between the inhibitor to Cys113, the inhibitor-bound Cys113 acts like a molecular crowbar. It destabilizes the short a helix between Cys 113 and Lys117 (helix D, FIG. 5), causing the helix to unfold and Lys117 to break its cation-pi interaction to Trp73. This allows the Lys117 to flip into the solvent, making it accessible for ubiquitination. The ubiquitinated hPin1 is targeted to the proteasome and degraded. The inhibitor is depicted in orange, the hPin1 domains in blue and purple, the ubiquitin in yellow, and the proteasome in multi-color. Bottom panels, inhibitor acts as a molecular crowbar that opens-up and destabilizes Pin1, resulting in increased exposure of Lys 117 and subsequent proteasomal degradation.

FIGS. 10A-10C. DELFIA assay for Pin1 and chemical structure of the biotinylated D-peptide. FIG. 10A) DELFIA dose response curve for the binding of a biotinylated D-peptide to 6His-Pin1. FIG. 10B) Z' factor determination for DELFIA using 158F10 and DMSO as positive and negative controls, respectively, with each having 14 replicates. Z′ factor=0.82. FIG. 10C) The chemical structure of the biotinylated D-peptide used to develop the heterogeneous assay is also reported.

FIG. 11. ΔTₘ curves of Pin1 alone (left peak) and in the presence of D-peptide (right peak).

FIG. 12. Denaturation thermal shift data and Western Blot data of selected agents. BxPC3 cells were treated with 0.1% DMSO or 15 μM of the reported agents for 24 h and Pin1 levels were quantified. Except for 164A2, agents inducing negative ΔTₘ values<−5° C. also cause PIN1 degradation in cell.

FIG. 13. Assignment of backbone ¹⁵N, ¹H chemical shift perturbations upon binding of agent 164A9.

FIGS. 14A-14C. FIG. 14A) DELFIA of 158F10, FIG. 14B) ΔTₘ and FIG. 14C) western blot image showing degradation of Pin1 by 158F10 after 24 h in MIA PaCa-2 cells.

FIGS. 15A-15E. The agents also cause PIN1 degradation in the pancreatic cancer cell line BxPC3 (FIG. 15A), MiaPaca-2 (FIG. 15B), or PANC-1 (FIG. 15C). In the example, cells were treated with 0.1% DMSO or 15 μM of the reported agents for 24 h and PIN1 levels were quantified. Compounds 158H9 and 164A10 are potent PIN1 binders with IC₅₀ values of 21.5 and 4.1 nM, respectively (FIG. 15D). The agents also cause thermal destabilization of PIN1 with ΔTₘ values of −8.41 and −8.95° C., respectively (FIG. 15E).

FIG. 16. Shows in vitro displacement affinity in a DELFIA assay, PIN1 induced cellular degradation in 3 different cell lines, and cell killing in two cell lines for the compound 164B8. 164B8 retains its PIN1 binding affinity (DELFTA assay IC₅₀ 4.85 nM) and PIN1 degradation in cell (DC₅₀s˜500 nM in the pancreatic cancer cell lines tested. Cell killing is improved likely due to improved cell permeability associated with the N-methylation as shown for MiaPaca2 and KPC cell lines (EC₅₀ 8.4 μM and 5.3 μM, respectively.

### DETAILED DESCRIPTION

Described herein include a ligand design strategy based on the selection of potent protein binders that can also induce target instability in vitro. In turn, these agents cause target degradation in cells. Application of this strategy to the proline cis-trans isomerase Pin1 resulted in potent compounds that are effective in inhibiting Pin1 and/or causing Pin1 degradation in several human cancer cell lines and that can be translated into potential anticancer agents. The design strategy of such agents, termed herein molecular crowbars, represents an efficient way to induce protein degradation in cell without the need of designing chimeric bi-dentate agents such as protein targeted chimeras (PROTACs) or molecular glues, hence could find wide applications in pharmacology and drug discovery. The invention also provides bi-dentate agents (i.e., conjugates of formula (I) wherein R² is —C(═O)-L-D).

### Definitions

The following definitions are used, unless otherwise described: halo or halogen is fluoro, chloro, bromo, or iodo. Alkyl, alkoxy, alkenyl, alkynyl, etc. denote both straight and branched groups; but reference to an individual radical such as propyl embraces only the straight chain radical, a branched chain isomer such as isopropyl being specifically referred to.

The term “alkyl”, by itself or as part of another substituent, means, unless otherwise stated, a straight or branched chain hydrocarbon radical, having the number of carbon atoms designated (i.e., C₁₋₈ means one to eight carbons). Examples include (C₁-C₈)alkyl, (C₂-C₈)alkyl, (C₁-C₆)alkyl, (C₁-C₃)alkyl, (C₂-C₆)alkyl and (C₃-C₆)alkyl. Examples of alkyl groups include methyl, ethyl, n-propyl, iso-propyl, n-butyl, t-butyl, iso-butyl, sec-butyl, n-pentyl, n-hexyl, n-heptyl, n-octyl, and higher homologs and isomers.

The term “alkenyl” refers to an unsaturated alkyl radical having one or more double bonds. Examples of such unsaturated alkyl groups include vinyl, 2-propenyl, crotyl, 2-isopentenyl, 2-(butadienyl), 2,4-pentadienyl, 3-(1,4-pentadienyl) and the higher homologs and isomers.

The term “alkynyl” refers to an unsaturated alkyl radical having one or more triple bonds. Examples of such unsaturated alkyl groups ethynyl, 1- and 3-propynyl, 3-butynyl, and higher homologs and isomers.

The term “alkoxy” refers to the formula-OR or radical thereof, where R is an alkyl as defined.

The term “alkyl-alkoxy” refers to an alkyl group in which one or more hydrogen atom has been replaced with an alkoxy group as defined above. Non-limiting examples of alkyl-alkoxy groups include, but are not limited to, —CH₂—OCH₃, —(CH₂)₂—OCH₃, or —CH₂—OCH₂CH₃, and the like.

The term “cycloalkyl” or “carbocycle” refers to a saturated or partially unsaturated (non-aromatic) all carbon ring having 3 to 8 carbon atoms (i.e., (C₃-C₈) carbocycle). The term also includes multiple condensed, saturated all carbon ring systems (e.g., ring systems comprising 2, 3 or 4 carbocyclic rings). Accordingly, carbocycle includes multicyclic carbocyles such as a bicyclic carbocycles (e.g., bicyclic carbocycles having about 3 to 15 carbon atoms, about 6 to 15 carbon atoms, or 6 to 12 carbon atoms such as bicyclo[3.1.0]hexane and bicyclo[2.1.1]hexane), and polycyclic carbocycles (e.g tricyclic and tetracyclic carbocycles with up to about 20 carbon atoms). The rings of the multiple condensed ring system can be connected to each other via fused, spiro and bridged bonds when allowed by valency requirements. For example, multicyclic carbocyles can be connected to each other via a single carbon atom to form a spiro connection (e.g., spiropentane, spiro[4,5]decane, etc), via two adjacent carbon atoms to form a fused connection (e.g., carbocycles such as decahydronaphthalene, norsabinane, norcarane) or via two non-adjacent carbon atoms to form a bridged connection (e.g., norbornane, bicyclo[2.2.2]octane, etc). Non-limiting examples of cycloalkyls include cyclopropyl, cyclobutyl, cyclopentyl, cyclohexyl, bicyclo[2.2.1]heptane, pinane, and adamantane.

The term “halo” or “halogen” refers to bromo, chloro, fluoro or iodo. In some embodiments, halogen refers to chloro or fluoro.

The term “aryl” as used herein refers to a single all carbon aromatic ring or a multiple condensed all carbon ring system wherein at least one of the rings is aromatic. For example, in certain embodiments, an aryl group has 6 to 20 carbon atoms, 6 to 14 carbon atoms, 6 to 12 carbon atoms, or 6 to 10 carbon atoms. Aryl includes a phenyl radical. Aryl also includes multiple condensed carbon ring systems (e.g., ring systems comprising 2, 3 or 4 rings) having about 9 to 20 carbon atoms in which at least one ring is aromatic and wherein the other rings may be aromatic or not aromatic (i.e., cycloalkyl). The rings of the multiple condensed ring system can be connected to each other via fused, spiro and bridged bonds when allowed by valency requirements. It is to be understood that the point of attachment of a multiple condensed ring system, as defined above, can be at any position of the ring system including an aromatic or a carbocycle portion of the ring. Non-limiting examples of aryl groups include, but are not limited to, phenyl, indenyl, indanyl, naphthyl, 1, 2, 3, 4-tetrahydronaphthyl, anthracenyl, and the like.

The term “heterocycle” or “heterocycloalkyl” refers to a single saturated or partially unsaturated ring that has at least one atom other than carbon in the ring, wherein the atom is selected from the group consisting of oxygen, nitrogen and sulfur; the term also includes multiple condensed ring systems that have at least one such saturated or partially unsaturated ring, which multiple condensed ring systems are further described below. Thus, the term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The sulfur and nitrogen atoms may also be present in their oxidized forms. Exemplary heterocycles include but are not limited to azetidinyl, tetrahydrofuranyl and piperidinyl. The term “heterocycle” or “heterocycloalkyl” also includes multiple condensed ring systems (e.g., ring systems comprising 2, 3 or 4 rings) wherein a single heterocycle ring (as defined above) can be condensed with one or more groups selected from cycloalkyl, aryl, and heterocycle to form the multiple condensed ring system. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heterocycle) can be at any position of the multiple condensed ring system including a heterocycle, aryl and carbocycle portion of the ring. In one embodiment the term heterocycle includes a 3-15 membered heterocycle. In one embodiment the term heterocycle includes a 3-10 membered heterocycle. In one embodiment the term heterocycle includes a 3-8 membered heterocycle. In one embodiment the term heterocycle includes a 3-7 membered heterocycle. In one embodiment the term heterocycle includes a 3-6 membered heterocycle. In one embodiment the term heterocycle includes a 4-6 membered heterocycle. In one embodiment the term heterocycle includes a 3-10 membered monocyclic or bicyclic heterocycle comprising 1 to 4 heteroatoms. In one embodiment the term heterocycle includes a 3-8 membered monocyclic or bicyclic heterocycle heterocycle comprising 1 to 3 heteroatoms. In one embodiment the term heterocycle includes a 3-6 membered monocyclic heterocycle comprising 1 to 2 heteroatoms. In one embodiment the term heterocycle includes a 4-6 membered monocyclic heterocycle comprising 1 to 2 heteroatoms. Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, 1,4-benzodioxanyl, spiro[cyclopropane-1,1′-isoindolinyl]-3′-one, isoindolinyl-1-one, 2-oxa-6-azaspiro[3.3]heptanyl, imidazolidin-2-one imidazolidine, pyrazolidine, butyrolactam, valerolactam, imidazolidinone, hydantoin, dioxolane, phthalimide, and 1,4-dioxane.

The term “heteroaryl” as used herein refers to a single aromatic ring that has at least one atom other than carbon in the ring, wherein the atom is selected from the group consisting of oxygen, nitrogen and sulfur; “heteroaryl” also includes multiple condensed ring systems that have at least one such aromatic ring, which multiple condensed ring systems are further described below. Thus, “heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. Exemplary heteroaryl ring systems include but are not limited to pyridyl, pyrimidinyl, oxazolyl or furyl. “Heteroaryl” also includes multiple condensed ring systems (e.g., ring systems comprising 2, 3 or 4 rings) wherein a heteroaryl group, as defined above, is condensed with one or more rings selected from cycloalkyl, aryl, heterocycle, and heteroaryl. It is to be understood that the point of attachment for a heteroaryl or heteroaryl multiple condensed ring system can be at any suitable atom of the heteroaryl or heteroaryl multiple condensed ring system including a carbon atom and a heteroatom (e.g., a nitrogen). Exemplary heteroaryls include but are not limited to pyridyl, pyrrolyl, pyrazinyl, pyrimidinyl, pyridazinyl, pyrazolyl, thienyl, indolyl, imidazolyl, triazolyl, tetrazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, and quinazolyl.

The terms “treat” and “treatment” refer to both therapeutic treatment and prophylactic or preventative measures, wherein the object is to prevent or slow down (lessen) an undesired physiological change or disorder. For example, the onset of a disorder or disease is prevented or delayed. The progression of a disease is slowed or stopped.

The phrase “therapeutically effective amount” means an amount of a compound of the present invention that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “mammal” as used herein refers to, e.g., humans, higher non-human primates, rodents, cows, horses, pigs, sheep, dogs and cats. In one embodiment, the mammal is a human.

The term “residue” as it applies to the residue of an E3 ligase targeting drug moiety refers to an E3 ligase targeting drug moiety that has been modified in any manner which results in the creation of an open valence. The open valence can be created by the removal of 1 or more atoms from the compound (e.g., removal of a single atom such as hydrogen or removal of more than one atom such as a group of atoms including but not limited to an amine, hydroxyl, methyl, amide (e.g., —C(═O)NH₂) or acetyl group). The open valence can also be created by the chemical conversion of a first function group of the compound to a second functional group of the compound (e.g., reduction of a carbonyl group, replacement of a carbonyl group with an amine) followed by the removal of 1 or more atoms from the second functional group to create the open valence.

As used herein, the term “PIN1” or “hPIN1” refers to human Peptidylprolyl Cis/Trans Isomerase, NIMA-Interacting 1 (also see NCBI accession number AAC50492). In certain embodiments, the compound of Formula I is a covalent inhibitor of PIN1. In certain embodiments, the compound of Formula I is capable of binding covalently to the catalytic Cys113 residue of PIN1's active site, or binding site.

The term “conjugate” as used herein refers to a compound of formula (I) wherein R² is —C(═O)-L-D.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention can contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

It will be appreciated by those skilled in the art that certain compounds described herein have a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase).

When a bond in a compound formula herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95% the absolute stereoisomer depicted. In another embodiment, the compound may be at least 99% the absolute stereoisomer depicted.

### E3 Ligase Targeting Drug Moiety (D)

The E3 ligase targeting drug moiety can be directed to any E3 ligase, including but not limited to cereblon, VHL, N-degrons such as for example UBR1, UBR2, UBR4, and UBR5, or E3 ligases such as SIAH or IAPs, and others.

The E3 ligase targeting drug moiety can be bonded to the linker L at any synthetically feasible position on the E3 ligase targeting drug moiety, provided the resulting conjugate of formula (I) effectively targets the proline cis-trans isomerase Pin1.

In one embodiment, the E3 ligase targeting drug moiety is:

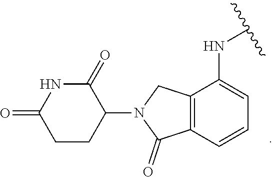 [SMILES: *Nc1cccc2c1CN(C1CCC(=O)NC1=O)C2=O]

### Linker (L)

As described herein, the E3 ligase targeting drug moiety can be bonded (connected) to the remainder of the conjugate of formula (I) through a linker (L). In one embodiment the linker is absent (e.g., the E3 ligase targeting drug moiety is bonded (connected) directly to the remainder of the conjugate of formula (I)). The linker can be variable provided the targeting conjugate functions as described herein. The linker can vary in length and atom composition and for example can be branched or non-branched or cyclic or a combination thereof. The linker may also modulate the properties of the targeted conjugate such as but not limited to solubility, stability and aggregation.

In one embodiment the linker comprises about 3-250 atoms. In one embodiment the linker comprises about 3-100 atoms. In one embodiment the linker comprises about 3-50 atoms. In one embodiment the linker comprises about 3-25 atoms.

In one embodiment the linker comprises about 10-250 atoms. In one embodiment the linker comprises about 10-100 atoms. In one embodiment the linker comprises about 10-50 atoms. In one embodiment the linker comprises about 10-25 atoms.

In one embodiment the linker comprises atoms selected from H, C, N, S, P and O.

In one embodiment the linker comprises atoms selected from H, C, N, S and O.

In one embodiment the linker comprises a branched or unbranched, saturated or unsaturated, hydrocarbon chain, having from about 1 to 25 (or 1-10, 1-5, 2-10, 2-5, 3-10, 3-5, 4-10, 4-5, or 5-10 carbon atoms) wherein one or more of the carbon atoms is optionally replaced independently by —O—, —S, —N(R^{a})₂, —N(R^{a})—, 3-7 membered heterocycle, 5-6-membered heteroaryl or carbocycle and wherein each chain, 3-7 membered heterocycle, 5-6-membered heteroaryl or carbocycle is optionally and independently substituted with one or more (e.g. 1, 2, 3, 4, 5 or more) substituents selected from (C₁-C₆)alkyl, (C₁-C₆)alkoxy, (C₃-C₆) cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, —N(R^{a})₂, hydroxy, oxo (═O), carboxy, aryl, aryloxy, heteroaryl, and heteroaryloxy, wherein each R^{a} is independently H or (C₁-C₆)alkyl.

In one embodiment, the linker comprises a ring that can be formed using click chemistry, for example:

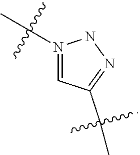

In one embodiment the linker comprises a branched or unbranched, saturated or unsaturated, hydrocarbon chain, having from about 1 to 25 (or 1-10, 1-5, 2-10, 2-5, 3-10, 3-5, 4-10, 4-5, or 5-10 carbon atoms), wherein one or more of the carbon atoms is optionally replaced independently by a ring that can be formed using click chemistry, —O—, —C(═O), —S, —N(R^{a})₂, or —N(R^{a})—, wherein each R^{a} is independently H or (C₁-C₆)alkyl.

In one embodiment the linker comprises a branched or unbranched, saturated or unsaturated, hydrocarbon chain, having from about 1 to 25 (or 1-10, 1-5, 2-10, 2-5, 3-10, 3-5, 4-10, 4-5, or 5-10 carbon atoms), wherein one or more of the carbon atoms is optionally replaced independently by —O—, —C(═O)—, —S, —N(R^{a})₂, or —N(R^{a})—, wherein each R^{a} is independently H or (C₁-C₆)alkyl.

In one embodiment the linker is linked to the remainder of the conjugate of formula (I) through a nitrogen atom.

In one embodiment the linker is linked to the E3 ligase targeting drug moiety through a carbonyl group.

In one embodiment the linker comprises a polyethylene glycol. In one embodiment the linker comprises a polyethylene glycol linked to the remainder of the conjugate of formula (I) through a nitrogen atom. In one embodiment the linker comprises a polyethylene glycol linked to the E3 ligase targeting drug moiety through a carbonyl group.

### Embodiments

In one embodiment, the compound or salt of Formula (I), is a compound or conjugate of Formula (Ia)

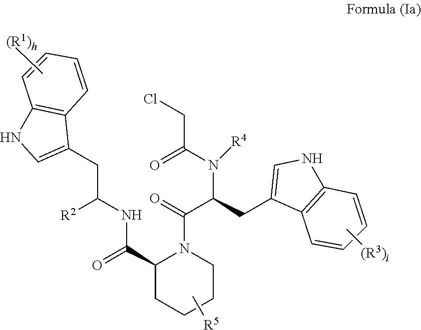

or a salt thereof.

In one embodiment, the compound or salt of Formula (I), is a compound or conjugate of Formula (Ib)

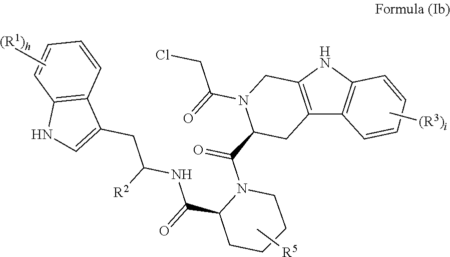

or salt thereof.

In one embodiment, each R¹ is independently halo, hydroxy, (C₁-C₆)alkyl, or (C₁—C₆)alkoxy.

In one embodiment, each R¹ is independently —Cl, —F, —CH₃, —C₂H5, —CH(CH₃)₂, —CH₂CH₂CH₃, —OCH₃, —OC₂H₅, —CN, —NO₂, —SO₂F, —OSO₂F, or —CH₂—O—CH₃.

In one embodiment, R² is H, (C₁-C₆)alkyl, aryl, heteroaryl, heterocycle, —C(═O)NH₂, or —C(═O)NH—Rₐ, wherein the (C₁-C₆)alkyl, aryl, heteroaryl, or heterocycle is optionally substituted with one or more substituents selected from the group consisting of halo, hydroxy, (C₁-C₆)alkyl, (C₁-C₆)alkoxy, —CN, —NO₂, —SO₂F, and —OSO₂F.

In one embodiment, R² is-C(═O)NH₂.

In one embodiment, R² is-C(═O)-L-D.

In one embodiment, each R³ is independently halo, hydroxy, (C₁-C₆)alkyl, or (C₁-C₆)alkoxy.

In one embodiment, each R³ is independently —Cl, —F, —CH₃, —C₂H₅, —CH(CH₃)₂, —CH₂CH₂CH₃, —OCH₃, —OC₂H₅, —CN, —NO₂, —SO₂F, —OSO₂F, or —CH₂—O—CH₃.

In one embodiment, h, i, and j are each independently 0, 1, or 2.

In one embodiment, R⁴ is H.

In one embodiment, R⁵ is absent.

In one embodiment, n is 0, and ring A is a (C₃-C₆) carbocycle ring.

In one embodiment, each R⁶ is independently halo, or (C₁-C₆)alkyl.

In one embodiment, the compound or salt is:

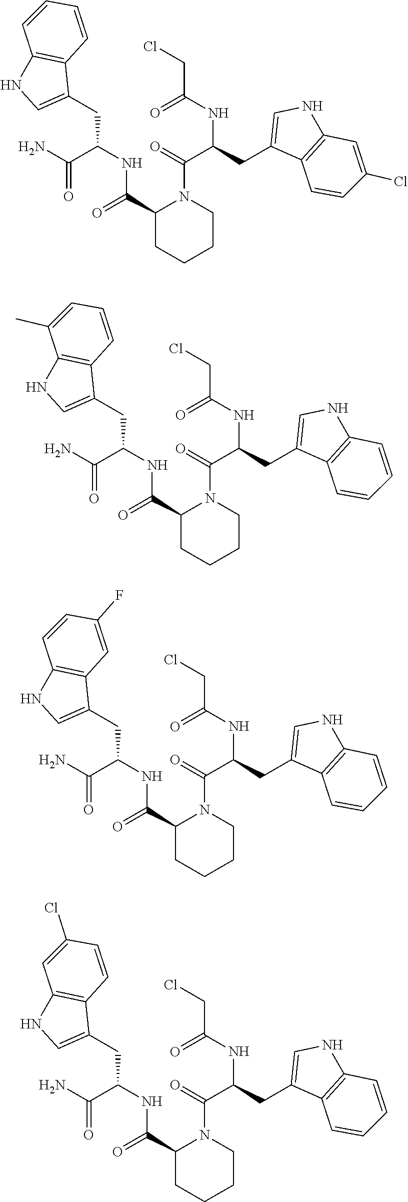 [SMILES: NC(=O)[C@H](Cc1c[nH]c2ccccc12)NC(=O)[C@@H]1CCCCN1C(=O)[C@H](Cc1c[nH]c2cc(Cl)ccc12)NC(=O)CCl ; Cc1cccc2c(C[C@H](NC(=O)[C@@H]3CCCCN3C(=O)[C@H](Cc3c[nH]c4ccccc34)NC(=O)CCl)C(N)=O)c[nH]c12 ; NC(=O)[C@H](Cc1c[nH]c2ccc(F)cc12)NC(=O)[C@@H]1CCCCN1C(=O)[C@H](Cc1c[nH]c2ccccc12)NC(=O)CCl ; NC(=O)[C@H](Cc1c[nH]c2cc(Cl)ccc12)NC(=O)[C@@H]1CCCCN1C(=O)[C@H](Cc1c[nH]c2ccccc12)NC(=O)CCl]

or salt thereof.

In one embodiment, the compound or salt is:

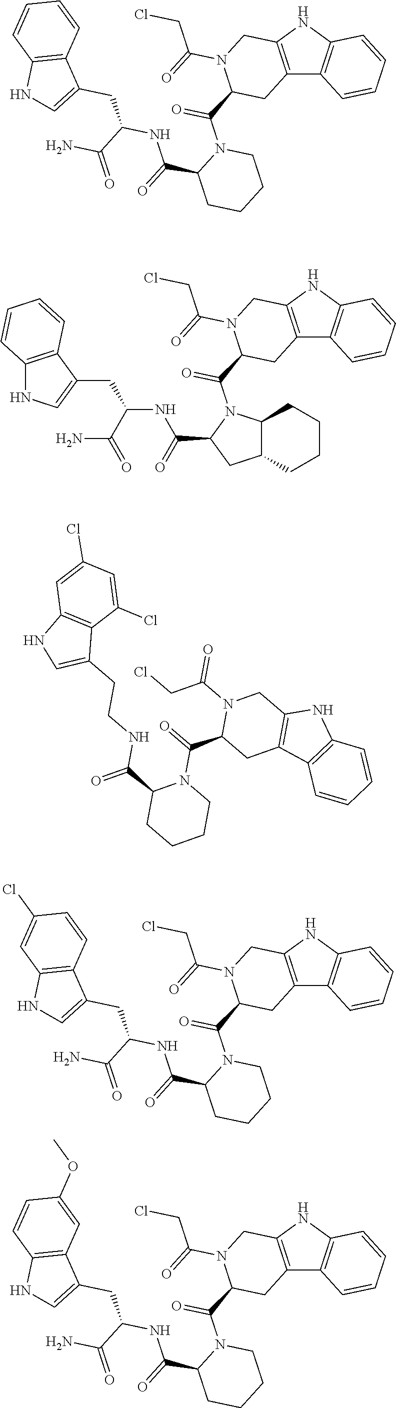 [SMILES: NC(=O)[C@H](Cc1c[nH]c2ccccc12)NC(=O)[C@@H]1CCCCN1C(=O)[C@@H]1Cc2c([nH]c3ccccc23)CN1C(=O)CCl ; NC(=O)[C@H](Cc1c[nH]c2ccccc12)NC(=O)[C@@H]1C[C@H]2CCCC[C@@H]2N1C(=O)[C@@H]1Cc2c([nH]c3ccccc23)CN1C(=O)CCl ; O=C(NCCc1c[nH]c2cc(Cl)cc(Cl)c12)[C@@H]1CCCCN1C(=O)[C@@H]1Cc2c([nH]c3ccccc23)CN1C(=O)CCl ; NC(=O)[C@H](Cc1c[nH]c2cc(Cl)ccc12)NC(=O)[C@@H]1CCCCN1C(=O)[C@@H]1Cc2c([nH]c3ccccc23)CN1C(=O)CCl ; COc1ccc2[nH]cc(C[C@H](NC(=O)[C@@H]3CCCCN3C(=O)[C@@H]3Cc4c([nH]c5ccccc45)CN3C(=O)CCl)C(N)=O)c2c1]

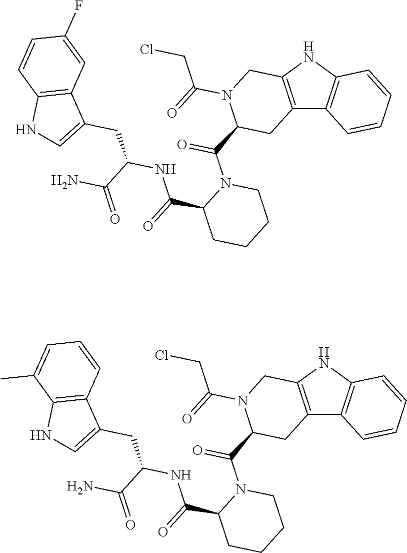 [SMILES: NC(=O)[C@H](Cc1c[nH]c2ccc(F)cc12)NC(=O)[C@@H]1CCCCN1C(=O)[C@@H]1Cc2c([nH]c3ccccc23)CN1C(=O)CCl ; Cc1cccc2c(C[C@H](NC(=O)[C@@H]3CCCCN3C(=O)[C@@H]3Cc4c([nH]c5ccccc45)CN3C(=O)CCl)C(N)=O)c[nH]c12]

or salt thereof.

In one embodiment, the compound or salt is selected from the group consisting of:

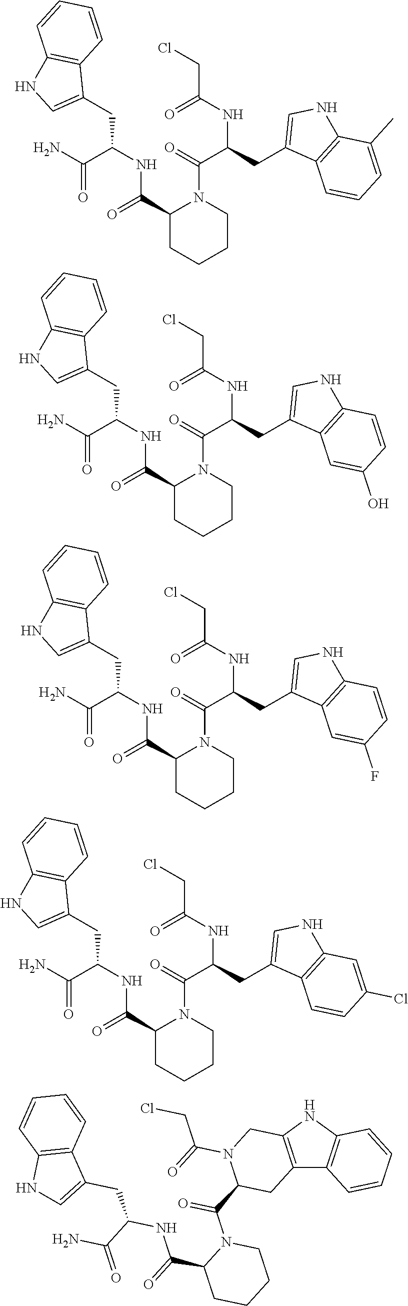

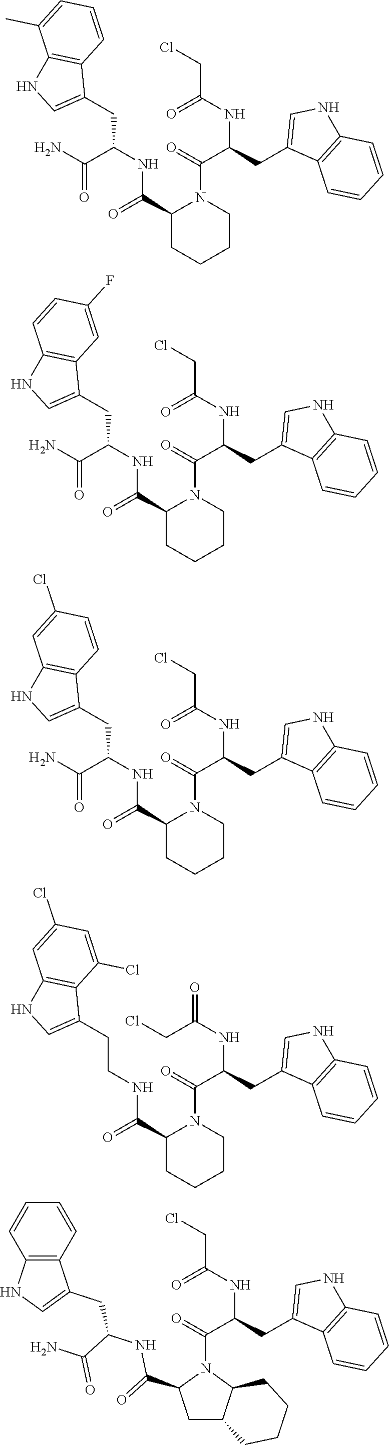

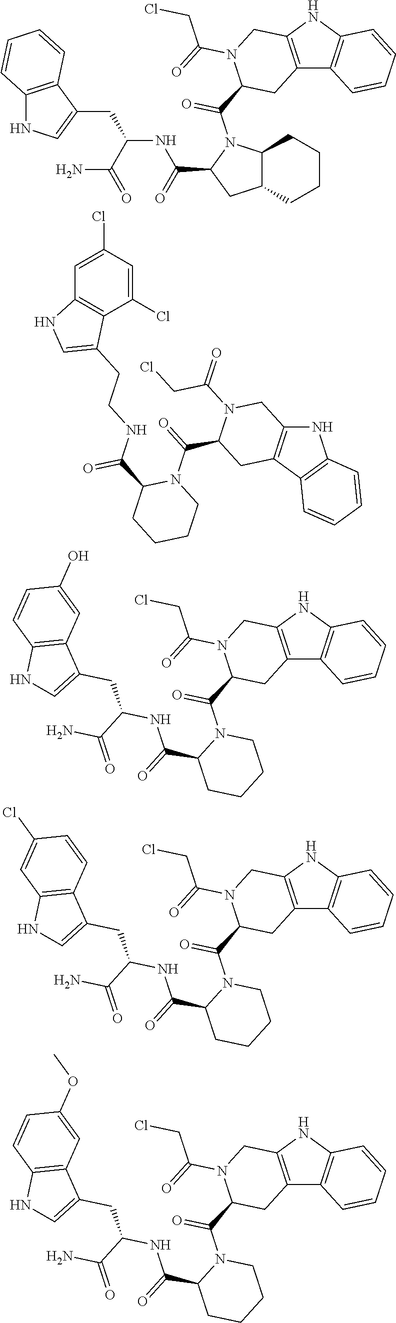

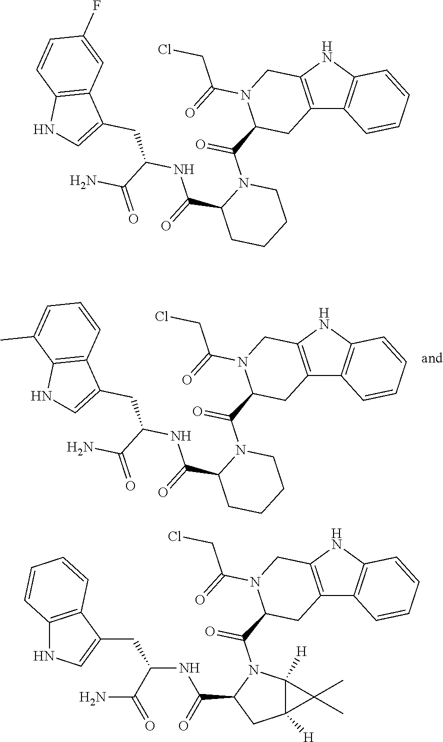

and salts thereof.

In one embodiment, the compound or salt is:

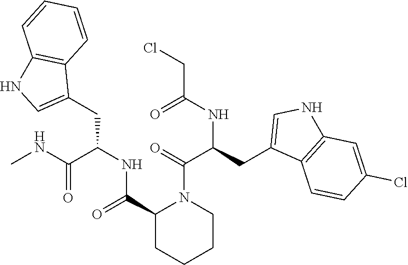 [SMILES: CNC(=O)[C@H](Cc1c[nH]c2ccccc12)NC(=O)[C@@H]1CCCCN1C(=O)[C@H](Cc1c[nH]c2cc(Cl)ccc12)NC(=O)CCl]

or a salt thereof.

In one embodiment, the compound or salt is selected from the group consisting of:

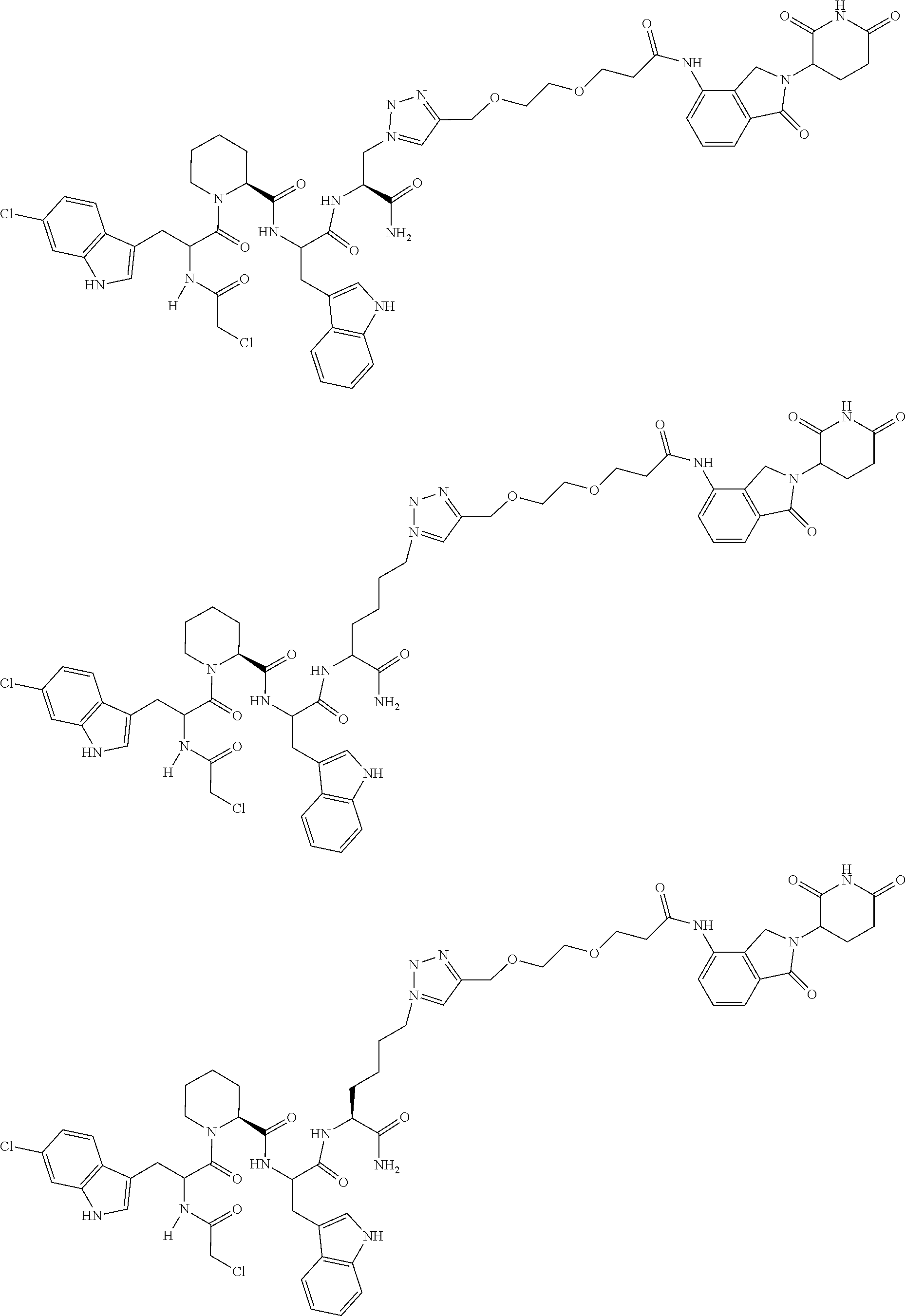

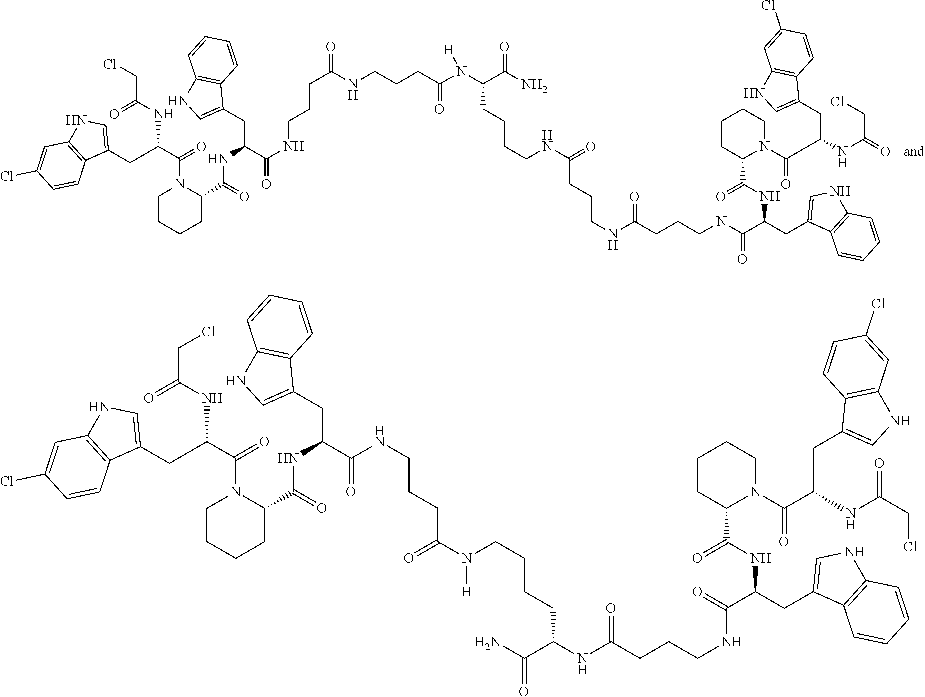

and salts thereof.

In one embodiment, the conjugate or salt is a conjugate of Formula (Ic)

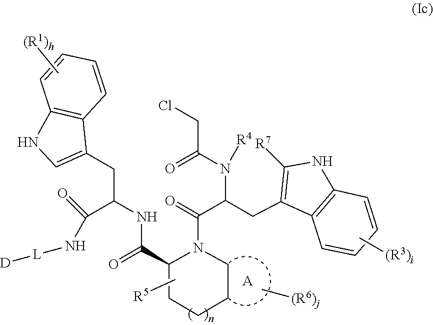

or a salt thereof, wherein:
L is absent or a linker;
D is the residue of an E3 ligase targeting drug moiety, the residue of another molecule targeting PIN1, or the residue of a ligand targeting a different protein target to induce its PIN1 driven degradation:
each R¹ is independently halo, hydroxy, (C₁-C₆)alkyl, (C₁-C₆)alkoxy, —CN, —NO₂, —SO₂F, —OSO₂F, or (C₁-C₆)alkyl-(C₁-C₆)alkoxy;
each R³ is independently halo, hydroxy, (C₁-C₆)alkyl, (C₁-C₆)alkoxy, —CN, —NO₂, —SO₂F, —OSO₂F, or (C₁-C₆)alkyl-(C₁-C₆)alkoxy;
h, i, and j are each independently 0, 1, 2, 3, or 4;
R⁴ is H, (C₁-C₆)alkyl, (C₂-C₆)alkenyl, or (C₂-C₆)alkynyl, and R⁷ is H; or R⁴ and R⁷ taken together are —CH₂— (i.e., R⁴ and R⁷ along with the intervening atoms form a 6-membered ring); R⁵ is absent, halo, (C₁-C₆)alkyl, (C₁-C₆)alkoxy, —CN, —NO₂, —SO₂F, —OSO₂F, (C₁-C₆)alkyl-(C₁-C₆)alkoxy, aryl, heteroaryl, or heterocycle;
n is 0 or 1;
ring A is absent or a (C₃-C₆) carbocycle ring;
each R⁶ is independently halo, hydroxy, (C₁-C₆)alkyl, (C₁-C₆)alkoxy, —CN, —NO₂, —SO₂F, or —OSO₂F; and
Rₐ is (C₁-C₆)alkyl, aryl, heteroaryl, or heterocycle, wherein the (C₁-C₆)alkyl, aryl, heteroaryl, or heterocycle is optionally substituted with one or more substituents selected from the group consisting of halo, hydroxy, (C₁-C₆)alkyl, (C₁-C₆)alkoxy, —CN, —NO₂, —SO₂F, and —OSO₂F.

In one embodiment, L comprises 3-100 atoms.

In one embodiment, L comprises about 10-100 atoms selected from H, C, N, S, P and O.

In one embodiment, L comprises a branched or unbranched, saturated or unsaturated, hydrocarbon chain, having from about 1 to 25 carbon atoms, wherein one or more of the carbon atoms is optionally replaced independently by —O—, —S, —N(R^{a})₂, —N(R^{a})—, 3-7 membered heterocycle, 5-6-membered heteroaryl or carbocycle and wherein each chain, 3-7 membered heterocycle, 5-6-membered heteroaryl or carbocycle is optionally and independently substituted with one or more (e.g. 1, 2, 3, 4, 5 or more) substituents selected from (C₁-C₆)alkyl, (C₁-C₆)alkoxy, (C₃-C₆) cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, —N(R^{a})₂, hydroxy, oxo (═O), carboxy, aryl, aryloxy, heteroaryl, and heteroaryloxy, wherein each R^{a} is independently H or (C₁-C₆)alkyl.

In one embodiment, L comprises a ring that can be formed using click chemistry.

In one embodiment, L comprises a branched or unbranched, saturated or unsaturated, hydrocarbon chain, having from about 1 to 25 carbon atoms, wherein one or more of the carbon atoms is optionally replaced independently by a group selected from the group consisting of a ring that can be formed using click chemistry, —O—, —C(═O)—, —S, —N(R^{a})₂, and —N(R^{a})—, wherein each R^{a} is independently H or (C₁-C₆)alkyl.

In one embodiment, L comprises a branched or unbranched, saturated or unsaturated, hydrocarbon chain, having from about 1 to 25 carbon atoms, wherein one or more of the carbon atoms is optionally replaced independently by a group selected from the group consisting of —O—, —C(═O)—, —S, —N(R^{a})₂, and —N(R^{a})—, wherein each R^{a} is independently H or (C₁-C₆)alkyl.

In one embodiment, L is selected from the group consisting of:

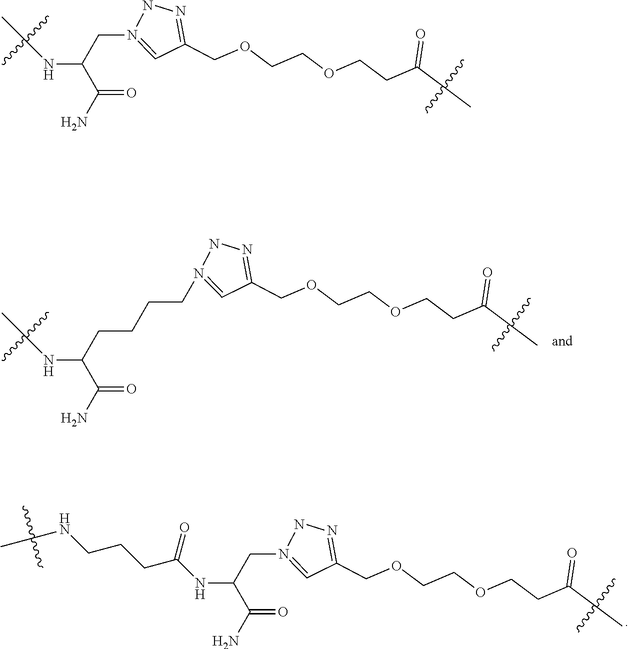

In one embodiment, L is selected from the group consisting of:

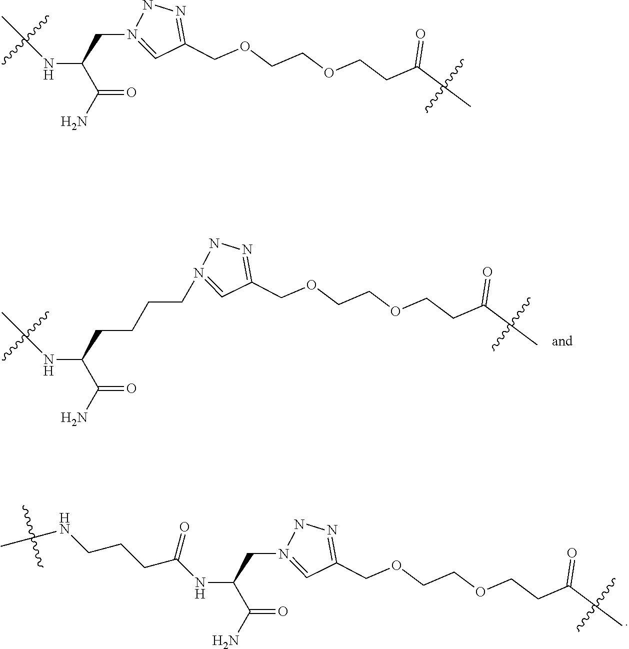

Assays for determining binding affinity or dissociation constant of a compound for a target protein are known in the art or described herein, including but not limited to, ligand displacement assay such as DELFIA. In certain embodiments, the compound of Formula I has a dissociation constant or IC₅₀ value that is lower than 2000 nM, 1500 nM, 1000 nM, 500 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, or less. In certain embodiments, the compound of Formula I has a dissociation constant or IC₅₀ that is lower than 500 nM. In certain embodiments, the compound of Formula I has a dissociation constant or IC₅₀ that is lower than 100 nM. In certain embodiments, the compound of Formula I has a dissociation constant or IC₅₀ that is lower than 50 nM. In certain embodiments, the compound of Formula I has a dissociation constant or IC₅₀ that is lower than 10 nM. In certain embodiments, the compound of Formula I has a dissociation constant or IC₅₀ that is in the range of 1-500 nM, 1-200 nM, 1-100 nM, 1-40 nM, 1-30 nM, 1-20 nM, or 1-10 nM. In certain embodiments, the compound of Formula I has a dissociation constant or IC₅₀ that is in the range of 1-50 nM, 2-40 nM, 3-30 nM or 4-20 nM.

In certain embodiments, the compound of Formula I is capable of causing thermal destabilization of PIN1. For example, protein thermal stability (e.g., melting temperature Tₘ of a protein, that is defined as the temperature at which 50% of the protein molecules are denatured) could be measured by a thermal shift assay known in the art or as described herein. In certain embodiments, compared to the native thermal stability (e.g., Tₘ) of PIN1 in the absence of the compound, the compound of formula I (once bound to PIN1) is capable of reducing the thermal stability (e.g., Tₘ) of PIN1.

In certain embodiments, the compound of Formula I is capable of reducing Tₘ of PIN1 by at least 5° C., 6° C., 7° C., 8° C., 9° C., 10° C., 11° C., 12° C., 13° C., 14° C., 15° C., 16° C., 17° C., 18° C., 19° C., 20° C. or more. In certain embodiments, the compound of Formula I is capable of reducing Tₘ of PIN1 by at least 5° C. In certain embodiments, the compound of Formula I is capable of reducing Tₘ of PIN1 by at least 10° C. Namely, in certain embodiments, the compound of Formula I is capable of causing a negative ΔTₘ by at least −5° C., −6° C., −7° C., −8° C., −9° C., −10° C., −11° C., −12° C., −13° C., −14° C., −15° C., −16° C., −17° C., −18° C., −19° C., −20° C. or more. In certain embodiments, the compound of Formula I is capable of causing a negative ΔTₘ by at least −5° C. in certain embodiments, the compound of Formula I is capable of causing a negative ΔTₘ by at least −10° C.

In certain embodiments, the compound of Formula I is capable of inducing cellular degradation of PIN1. In certain embodiments, the compound of Formula I is capable of decreasing the level of PIN1 in a cell that has been contacted with the compound.

In certain embodiments, the compound of Formula I has a DC₅₀ value (the concentration at which 50% cellular PIN1 degradation was observed) that is lower than 15 μM, 10 μM, 5 μM, 1 μM, 500 nM, 400 nM, 300 nM, 200 nM, 100 nM, or less. In certain embodiments, the compound of Formula I has a DC₅₀ that is lower than 5 μM. In certain embodiments, the compound of Formula I has a DC₅₀ that is lower than 1 μM. In certain embodiments, the compound of Formula I has a DC₅₀ that is lower than 500 nM.

Without wanting to be bound by theory, a compound's ability to reduce a target protein thermal stability may be correlated with the compound's ability to induce the target protein's degradation within cells. For example, in certain embodiments, the compound (e.g., once bound to a target protein such as PIN1) is capable of facilitating the unfolding of the target protein, the exposure of certain residue(s) (e.g., Lys 117 of PIN1), and/or the ubiquitination and proteosome degradation of the target protein in cells.

### Certain Methods

Certain embodiments of the invention provide a method of inhibiting and/or degrading PIN1 in vitro or in vivo, comprising contacting PIN1 with a compound of Formula I. In certain embodiments, the contacting is conducted in vitro. In certain embodiments, the contacting is conducted in vivo.

Certain embodiments of the invention provide a method of treating a PIN1 associated disease in a mammal in need thereof, comprising administering a therapeutically effective amount of a compound of Formula I, to the mammal. In certain embodiments, the PIN1 associated disease is related to PIN1 overexpression or higher activity of PIN1 as compared to a reference level in normal cells/tissue of a healthy mammal. In certain embodiments, the PIN1 associated disease is cancer. For example, the compound described herein could target or inhibit cancer cells and/or cancer associated fibroblasts. As used herein, the term “cancer associated fibroblasts” refers to fibroblasts located within the tumor matrix or surrounding the tumor such as located at the fibrous capsule or at the boundary between the tumor and neighboring tissue. In certain embodiments, the cancer is pancreatic cancer, breast cancer, prostate cancer, non-small cell lung cancer, colon cancer, ovarian cancer, endometrial cancer, head and neck cancer, or melanoma. In certain embodiments, the cancer is a KRAS activating mutation related cancer. In certain embodiments, the cancer cells comprise a KRAS activating mutation selected from the group consisting of KRAS (G12C), KRAS (G12D), KRAS (G12S), and KRAS (G13D).

Compounds of the invention can also be administered in combination with other therapeutic agents, for example, other agents that are useful for the treatment of cancer. In certain embodiments, the method of treating further comprises administering a second therapeutic agent to the mammal. Examples of such agents include chemotherapeutic agents (e.g., gemcitabine, Abraxane, oxaliplatin, fluorouracil, folinic acid, or combination thereof, such as FOLFOX), kinase inhibitors (e.g., Erlotinib), KRAS inhibitors (e.g., Sotorasib), or immune checkpoint inhibitors (e.g., anti-PD-1 or anti-PD-L1 antibody)

Certain embodiments of invention provide a compound described herein or a pharmaceutically acceptable salt thereof, for treating a PIN1 associated disease.

Certain embodiments of invention provide a compound described herein or a pharmaceutically acceptable salt thereof, for use in medical therapy.

Certain embodiments of invention provide the use of a compound described herein or a pharmaceutically acceptable salt thereof to prepare a medicament for treating a PIN1 associated disease in a mammal.

Certain embodiments of invention also provide a method of developing degrader compound for a target protein, comprising
contacting a test compound with the target protein in vitro,
determining the binding affinity of the test compound for the target protein,
determining the thermal stability of the target protein in the absence and presence of the test compound,
identifying the test compound as a degrader compound wherein the test compound is determined to be capable of binding the target protein and is determined to be capable of reducing the thermal stability of the target protein.

In certain embodiments, determining the binding affinity comprises conducting a dissociation-enhanced lanthanide fluorescent immunoassay (DELFIA), or isothermal titration calorimetry.

In certain embodiments, determining the binding affinity comprises determining the dissociation constant for the target or IC₅₀ (e.g., in a ligand displacement assay).

In certain embodiments, the dissociation constant or IC₅₀ is <1000 nM, 500 nM, 300 nM, 200 nM, 100 nM, 90 nM, 80 nM, 70 nM, 60 nM, 50 nM, 40 nM, 30 nM, 20 nM, or 10 nM. In certain embodiments, the dissociation constant or IC₅₀ is <500 nM. In certain embodiments, the dissociation constant or IC₅₀ is <100 nM. In certain embodiments, the dissociation constant or IC₅₀ is <50 nM. In certain embodiments, the dissociation constant or IC₅₀ is <10 nM.

In certain embodiments, determining the thermal stability comprises conducting a thermal shift assay, e.g., wherein the melting temperature (Tₘ) of the target protein is determined.

In certain embodiments, the test compound induces a Tₘ reduction of 5° C. or greater (e.g., a Tₘ reduction of 6° C., 7° C., 8° C., 9° C., 10° C., 11° C., 12° C., 13° C., 14° C., 15° C., 16° C., or greater).

### Composition and Administration

Certain embodiments of the invention also provide a composition comprising a compound as described, or a salt (e.g., pharmaceutically acceptable) thereof and a pharmaceutically acceptable carrier. Compounds described herein (including salt, solvate, stereoisomer or prodrug thereof) can be formulated as pharmaceutical compositions and administered to a mammalian host, such as a human patient in a variety of forms adapted to the chosen route of administration, i.e., orally or parenterally, by intravenous, intramuscular, intrathecal, topical, intranasal, inhalation, suppository, sub dermal osmotic pump, intraperitoneal, intradermal or subcutaneous routes.

Thus, the present compounds may be systemically administered, e.g., orally or intravenously, in combination with a pharmaceutically acceptable vehicle such as an inert diluent or a carrier (pharmaceutically acceptable excipients are well known in the field). The composition may be freeze-dried into lyophilized formulation (e.g., lyophilized cake), may be enclosed in hard or soft shell gelatin capsules, or may be compressed into tablets. For oral therapeutic administration, the active compound may be combined with one or more excipients and used in the form of ingestible tablets, buccal tablets, troches, capsules, elixirs, suspensions, syrups, wafers, and the like. The capsules, tablets or other oral delivery formulation may have enteric coating for controlled release of the compound at desired intestinal segment. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

Lyophilized formulations may also contain carrier such as bulking agent (e.g., mannitol or glycine) and cryoprotectant/lyoprotectant (e.g., trehalose or sucrose). Lyophilized formulation can be reconstituted into a liquid dosage form using saline, 5% dextrose solution or sterile water before administration. The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician, pharmacist, or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

Compounds of the invention described herein can also be administered in combination with other therapeutic agent(s). For example, compounds of the invention, or pharmaceutical salts thereof, may be administered with other agent(s) that are useful for treating cancer.

In one embodiment, the invention also provides a composition comprising a compound of formula I, or a pharmaceutically acceptable salt thereof, at least one other therapeutic agent, and a pharmaceutically acceptable diluent or carrier. The invention also provides a kit comprising a compound of the invention described herein, or a pharmaceutically acceptable salt thereof, and optionally at least one other therapeutic agent, packaging material, and instructions for administering the compound of the invention described herein or the pharmaceutically acceptable salt thereof and the other optional therapeutic agent or agents to an mammal to modulate PIN1 activity, and/or treat diseases associated with PIN1 (e.g., cancer).

The invention will now be illustrated by the following non-limiting Examples.

### EXAMPLES

### Example 1. Targeted Degradation of Pin1 by Protein Destabilizing Compounds

The concept of targeted protein degradation (TPD) is at the forefront of modern drug discovery, which aims to eliminate disease-causing proteins using specific molecules. In this Example 1, the idea to design protein degraders based on the section of ligands that cause protein destabilization, hence that facilitate the cellular breakdown of the target was explored. The studies herein present covalent agents targeting Pin1, a cis-trans prolyl isomerase that plays an important role in tumorigenesis. The design strategy in this Example involved iterative optimizations of agents for potency and Pin1 destabilization in vitro. Biophysical and cellular studies suggest that the agents act like molecular crowbars, displacing protein-stabilizing interactions that open the structure for recognition by the ubiquitin-proteasome degradation machinery. This approach resulted in a series of potent and effective Pin1 degraders with potential applications in target validation and in therapeutic development. This design strategy can identify molecular degraders without engineering bifunctional agents that artificially create interactions between a disease-causing protein and a ubiquitin ligase.

### INTRODUCTION

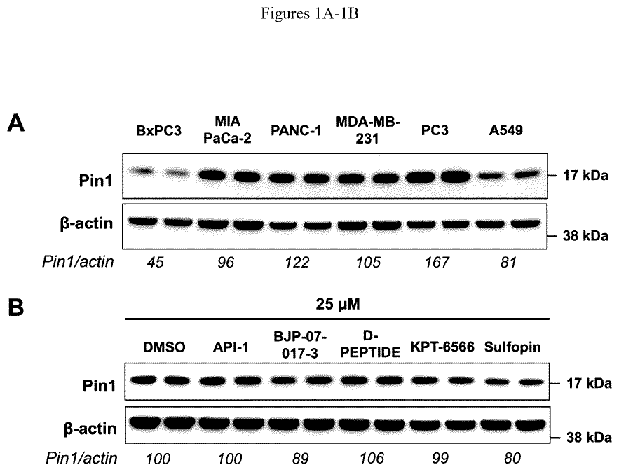

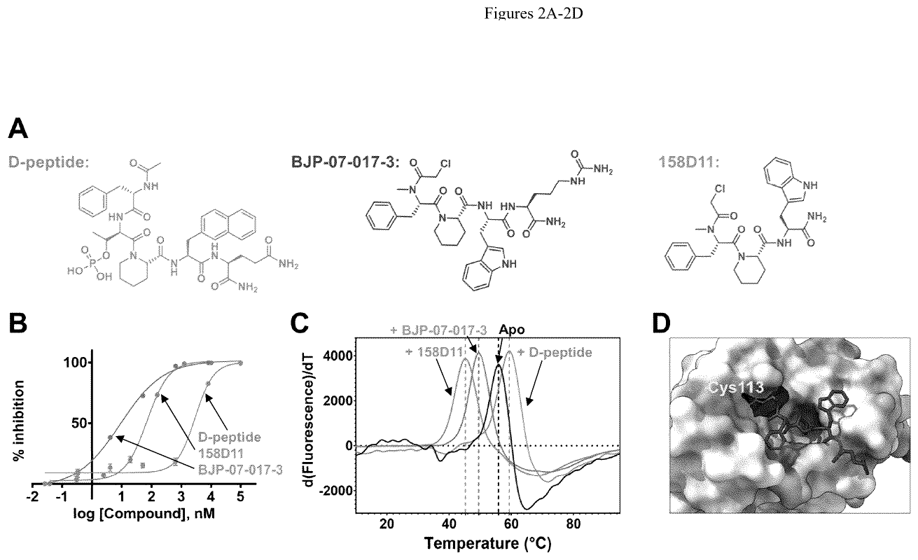

Proline-directed phosphorylation is a signaling event that is central to the activation of several cancer mechanisms, regulating both oncoproteins and tumor suppressors. These signaling events are further regulated by the proline isomerase Pin1 that recognizes specific pSer-Pro or pThr-Pro motifs. (Lu Z & Hunter T (2014) Cell Res 24(9):1033-1049; Sun Q, et al. (2020) Cell Prolif 53(5):e12816; Wu W, et al., (2022) Pharmacol Res 184:106456; and Zhou X Z & Lu K P (2016) Nat Rev Cancer 16(7):463-478). Pin1 overexpression is a major contributor to tumorigenesis, activating several oncoproteins, including proteins in the KRAS pathway, (5) and simultaneously inactivating several tumor suppressors (Lu Z & Hunter T (2014) Cell Res 24(9):1033-1049; and Zhou X Z & Lu K P (2016) Nat Rev Cancer 16(7):463-478). Pin1 has also been reported to confer chemotherapy-resistance in pancreatic cancers, by causing the degradation of the gemcitabine uptake-transporter, ENT1 (equilibrative nuclear transporter-1) both in tumor cells and cancer-associated fibroblasts (Koikawa K, et al. (2021) Cell 184(18):4753-4771 e4727; and Liu J, et al. (2022) Nat Commun 13(1):4308). Hence, the development of Pin1 inhibitors could increase the sensitivity of cancer cells to both chemotherapy and immunotherapy (Koikawa K, et al. (2021) Cell 184(18):4753-4771 e4727), and could find similar applications in a variety of tumors. For these reasons, several direct attempts at obtaining drug-like small molecule inhibitors from both pharmaceutical companies and academic laboratories are ongoing. Small molecule inhibitors were reported that nonetheless often contain a phosphate or carboxylate as an isostere of the pThr, which seemed to be required to maintain a basic affinity for the critical phosphate-binding pocket of the Pin1 protein (Guo C, et al. (2009) Bioorg Med Chem Lett 19(19):5613-5616; and Guo C, et al. (2014) Bioorg Med Chem Lett 24(17):4187-4191). Pfizer has identified an inhibitor that repressed the PPIase activity of Pin1 at nanomolar concentrations in vitro by using a structure-based design strategy and the crystal structure of Pin1 (Guo C, et al. (2009) Bioorg Med Chem Lett 19(19):5613-5616; and Guo C, et al. (2014) Bioorg Med Chem Lett 24(17):4187-4191). Vernalis identified a small molecule using a fragment-based approach to derive Pin1 inhibitors through an in vitro enzyme assay (Potter A J, et al. (2010) Bioorg Med Chem Lett 20(2):586-590). However, these attempts were overall unsuccessful as the resulting agents were poorly active or inactive in cell lines, likely due to the phosphate or carboxylate that renders the inhibitors poorly permeable, or because the enzyme is very efficient, and it is difficult to attain complete inhibition with reversible inhibitors. For this reason, more recent attempts were targeting binding site Cys113 to obtain irreversible inhibitors. For example, the agent KPT-6566 (see below), a Pin1 small molecule inhibitor, was identified from a large collection of commercial compounds (Campaner E, et al. (2017) Nat Commun 8:15772). Structurally, the electrophile sulfonyl-acetate moiety of KPT-6566 directly faces the nucleophile sulfur atom of Cys113. Interestingly, covalent inhibition by KPT-6566 also promoted degradation of Pin1, which should render the agents significantly more effective (Campaner E, et al. (2017) Nat Commun 8:15772). However, KPT-6566 presents poor drug-like characteristics, and it is likely that it may have a variety of unpredictable off-target effects in vivo (Campaner E, et al. (2017) Nat Commun 8:15772) These observations stimulated additional research in very recent years using more rational approaches including structure-based design starting from substrate peptide mimetics (Xu G G & Etzkorn F A (2009) Drug News Perspect 22(7):399-407; and Wildemann D, et al. (2006) J Med Chem 49(7):2147-2150). These agents have moderate to high activity in vitro but often are inactive in cell, or require cell permeabilizing moieties (Liu T, Liu Y, Kao H Y, & Pei D (2010) J Med Chem 53(6):2494-2501), hence generally lack the drug-like properties necessary to effectively translate these agents in possible therapeutics (Wildemann D, et al. (2006) J Med Chem 49(7):2147-2150). Combining these observations, very recently two new series of Cys113 covalent agents have been reported (Dubiella C, et al. (2021) Nat Chem Biol 17(9):954-963; and Pinch B J, et al. (2020) Nat Chem Biol 16(9):979-987). One is based on the structure of D-peptide (FIGS. 1-2) but containing a chloro-acetamide designed to be juxtaposed to Pin1 Cys113 to create a covalent adduct (Pinch B J, et al. (2020) Nat Chem Biol 16(9):979-987). However, due to its peptide nature, the agent is not particularly active in cell (see below). A second attempt deployed a screening of a large library of Cys targeting agents, that resulted in the identification of a small molecule, sulfopin, that apparently selectively targets Cys113 in vitro and in cell (Dubiella C, et al. (2021) Nat Chem Biol 17(9):954-963). However, sulfopin causes Pin1 degradation only at higher concentrations in cell (see below) (Dubiella C, et al. (2021) Nat Chem Biol 17(9):954-963).

In these studies, Pin1 Cys113 covalent agents with increased affinity in vitro that can more effectively cause Pin1 degradation in cell were sought. Iterative design and structure activity relations studies were guided by not only biochemical displacement assays, to assess for potency, but also by measurements of denaturation thermal shift induced by test agents, whereby it was observed that covalent compounds that can destabilize Pin1 in vitro also resulted more effective in causing Pin1 degradation in cell. Using this innovative strategy, agents that can bind to Pin1 in the low nanomolar range (IC₅₀s 4-20 nM range) and cause effective Pin1 degradation in various human cancer cell lines (DC₅₀s<500 nM) were identified. The molecular basis for the activity of the compounds as determined by solution NMR spectroscopy measurements, X-ray crystallography, and mutagenesis studies, suggested that the agents may act as molecular crowbars, inducing conformational changes that destabilize the protein and presumably lead to recognition of the partially unfolded protein by the ubiquitin-proteosome degradation system in cell. Hence, the reported agents represent not only potent Pin1-targeting degrading compounds, but this approach may represent an innovative design strategy to obtain targeted protein degraders.

### Results

### Assays and Design Strategy for the Identification and Iterative Optimization of Pin1 Degraders

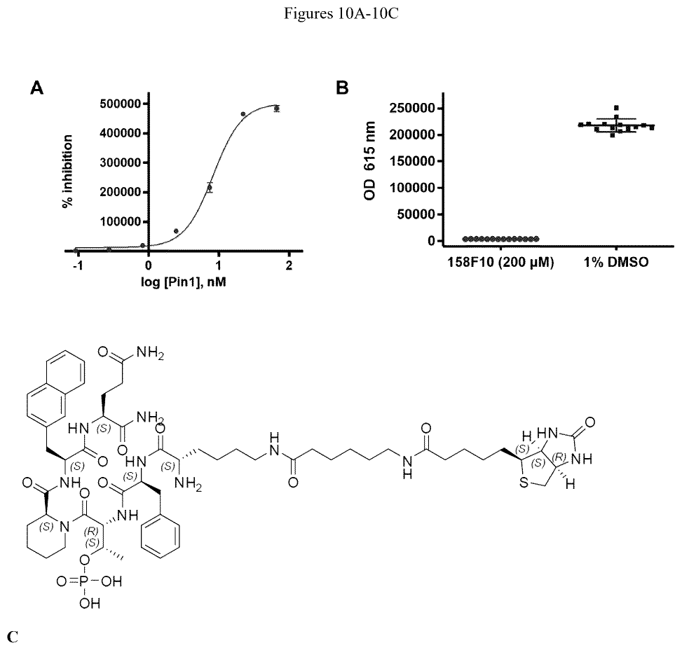

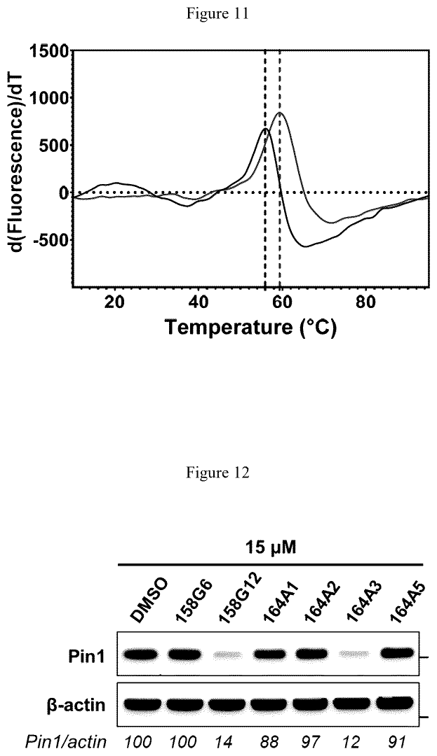

Important to the success of structure activity relations studies, robust biochemical, biophysical, and cell-based assays were implemented that enabled rapid and iterative evaluations of test Pin1 targeting agents. First, the DELFIA (Dissociation-enhanced lanthanide fluorescent immunoassay) assay platform was used to derive a robust heterogeneous assay to monitor the ability of test agents to displace the binding between Pin1 and the D-Peptide (Table 1), which was obtained in a biotinylated form (FIG. 10). This assay platform produces highly reproducible data and it is superior to biochemical prolyl cis-trans isomerization assay surrogates, or other homogeneous displacement assays such as the florescence polarization (Pinch B J, et al. (2020) Nat Chem Biol 16(9):979-987) that are more prone to false positives (Rega M F, Reed J C, & Pellecchia M (2007) Bioorg Chem 35(2):113-120). Second, for an orthogonal biophysical assay denaturation thermal shift measurements were used. Pin1 was incubated with vehicle control (buffer with 1% DMSO), or test agents at protein: ligand ratio of 1:2 (20 μM protein-40 μM compound) for 10 min at 10° C. in the presence of the fluorescent dye SYPRO Orange. The samples were gradually exposed to increasing temperature (linear gradient from 10° C. to 95° C.), causing the protein to unfold. This unfolding allowed the fluorescent dye to bind to the protein's exposed hydrophobic residues, resulting in an increase in fluorescence. The thermal shift assay measures the protein's melting temperature (Tₘ), which is the temperature at which the protein denatures by 50%. Ligands can affect a protein target to thermal denaturation, which could manifest in a positive or negative shift in the protein denaturation temperature (ΔTₘ) (Bai N, Roder H, Dickson A, & Karanicolas J (2019) Scientific Reports 9; and Kranz J K & Schalk-Hihi C (2011) Fragment-Based Drug Design: Tools, Practical Approaches, and Examples 493:277-298). Using these two assays, a series of commercially available Pin1 inhibitors were profiled (Table 1). The non-covalent agent D-peptide resulted only moderately active, with an IC₅₀ value of ˜3 μM that closely resembled the dissociation constant for this agent determined via isothermal titration calorimetry, and the ˜ 3 μM IC₅₀ value as reported by a fluorescence polarization assay (Pinch B J, et al. (2020) Nat Chem Biol 16(9):979-987). Typical of weaker, low micromolar affinity binders, a modest stabilization to thermal denaturation of Pin1 upon binding of the D-peptide with a ΔTₘ of 3.4° C. (Table 1) was observed.

TABLE 1

| | | | DELFIA | ΔTm (° C.) |
|---|---|---|---|---|
| Name | Structure | MW | IC₅₀ (nM) | 30 min |
| 158A1 (D- peptide) | 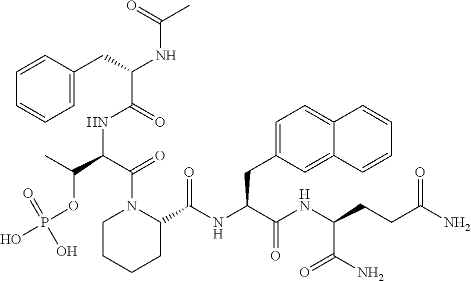 | 823.33 | 2912 ± 103 | 3.40 ± 0.16 |
| 158A12 (BJP-07- 017-3) | 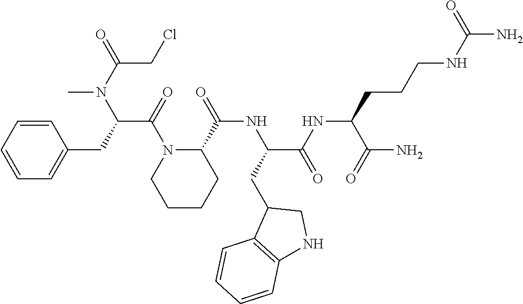 [SMILES: CN(C(=O)CCl)[C@@H](Cc1ccccc1)C(=O)N1CCCC[C@H]1C(=O)N[C@@H](CC1CNc2ccccc21)C(=O)N[C@@H](CCCNC(N)=O)C(N)=O] | 709.24 | 9 ± 1 | −6.40 ± 0.04 |
| Sulfopin | 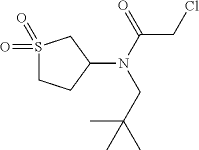 [SMILES: CC(C)(C)CN(C(=O)CCl)C1CCS(=O)(=O)C1] | 281.08 | 53 ± 10 | −0.64 ± 0.00 |
| API-1 | 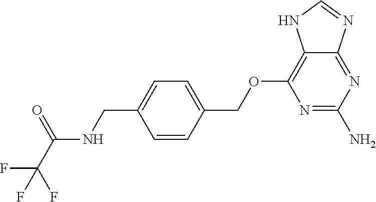 [SMILES: Nc1nc(OCc2ccc(CNC(=O)C(F)(F)F)cc2)c2[nH]cnc2n1] | 366.30 | >100 μM | −0.30 ± 0.32 |
| KPT- 6566 | 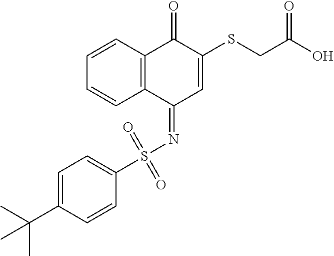 [SMILES: CC(C)(C)c1ccc(S(=O)(=O)/N=C2/C=C(SCC(=O)O)C(=O)c3ccccc32)cc1] | 443.54 | 189 ± 39 | −8.40 ± 0.08 * |

Chemical structures and characterization of five known Pin1 inhibitors (D-peptide, BJP-07-017-3, Sulfopin, API-1, and KPT-6566). DELFIA results were performed with a 6-h pre-incubation followed by a 2-h incubation. Thermal shift conditions: Protein:compound 1:2, 20X Sypro Orange Dye, Buffer: 50 mM phosphate, pH = 7.5, 150 mM NaCl, 3% DMSO final; * In the analysis by thermal shift of Pin1 in the presence of KPT-6566, a minor peak, for which ΔTm value was −22.71 ± 0.19° C., was observed. In DELFIA assay, standard deviation values are obtained from duplicate measurements. In the thermal shift assay, standard errors are reported from four replicate measurements.

For the reported covalent agents such as sulfopin (Dubiella C, et al. (2021) Nat Chem Biol 17(9):954-963), BJP-07-017-3 (Pinch B J, et al. (2020) Nat Chem Biol 16(9):979-987), or KPT-6566 (Campaner E, et al. (2017) Nat Commun 8:15772), a 6-h pre-incubation period was included in the DELFIA assay to allow the covalent reaction to take place. For those agents, IC₅₀ values that fell in the low nanomolar range were observed, consistent with what was reported previously for those compounds. Putative Pin1 ligand API-1 displayed a very weak ability to displace the D-peptide (IC₅₀>100 μM), despite the reported 2.9 μM dissociation constant by isothermal titration calorimetric measurements (Pu W, et al. (2018) Hepatology 68(2):547-560), perhaps indicating that it may bind on a different site on the protein. Surprisingly, however, all covalent agents caused a decrease in thermal stability of Pin1 upon binding (Table 1), indicating that covalent modification of Cys113 caused conformational instability of the protein making it more sensitive to thermal denaturation. As mentioned above, sulfopin, BJP-07-017-3, and KPT-6566 have been reported to cause Pin1 degradation in cell, hence, the possibility that there is a correlation between ligand induced thermal instability and ligand induced Pin1 degradation in cell was investagated. To make those determinations in a broader panel of cancer cell lines three pancreatic cancer cell lines, BxPC3, MIA-PaCa-2, and PANC-1, the breast cancer cell line MDA-MB-231, the prostate cancer cell line PC3, and the non-small cell lung cancer cell line A549 were chosen. Each cell line represents various states of KRAS activating mutations (for example BxPC3 is wt-KRAS, while MIAPaCa2 carries the KRAS(G12C) mutation, and the PANC-1 the KRAS(G12D) (Miquel M, Zhang S, & Pilarsky C (2021) Front Cell Dev Biol 9:748631), A549 is KRAS(G12S) (Wang H, et al. (2019) EBioMedicine 49:106-117), MDA-MB-231 is KRAS(G13D) (Kim R K, et al. (2015) Exp Mol Med 47(1):e137), PC3 is wt-KRAS (Bouali S, et al. (2009) Oncol Rep 21(3):731-735). Pin1 protein levels in these cell lines were established using a specific Pin1 antibody in western blot analyses (FIG. 1A). The data confirmed that all these selected human cancer cell lines expressed a significant level of Pin1 (FIG. 1A).

Next, each cell line was exposed to a relatively high concentration of each of the Pin1 inhibitors from Table 1 and measured their ability to cause Pin1 degradation in the Bx-PC3 cell line under the experimental conditions reported in FIG. 1B. Exposing Bx-PC3 cell line to 25 μM of the individual Pin1 inhibitors for 24 h, resulted in a significant Pin1 degradation in cell with agents BJP-07-017-3 and sulfopin (FIG. 1B). It was hypothesized that this observed degradation in the series correlated with the increased sensitivity of Pin1 to thermal degradation induced by BJP-07-017-3 in vitro (Table 1). Based on this hypothesis and the developed assays more potent and effective Pin1 inhibitors were sought by designing agents that are not only potent in the DELFIA assay but also cause a more sizable and complete thermal destabilization of Pin1 in vitro.

### Design, Synthesis, and Characterization of PIN1 Destabilizing Agents.

Given the ability of BJP-07-017-3 to cause Pin1 thermal destabilization in vitro (ΔTₘ=−6.4° C., Table 1) and some Pin1 degradation in cell (FIG. 1B), it was hypothesized that the cellular degradation induced by the agent may be related to its ability to induce Pin1 thermal destabilization in vitro. Based on this hypothesis, a series of ligands were generated and their ability to bind Pin1 was characterized via the DELFIA displacement assay described above. The ability of each test agent to cause a negative denaturation thermal shift was also measured. Preliminary structure-activity relationships studies of the BJP-07-017-3 compound are reported in Table 2, which highlights the importance of the chloroacetamide that reacts with Cys113 (agent 158B3), of the citrulline residue at the C-terminus (agent 158D11), and of the N-methylation on the Phenylalanine residue (agent 158D10).

TABLE 2

| | | | DELFIA | |
|---|---|---|---|---|
| Name | Structure | MW | IC₅₀ (nM) | ΔTm (° C.) |
| 158B3 | 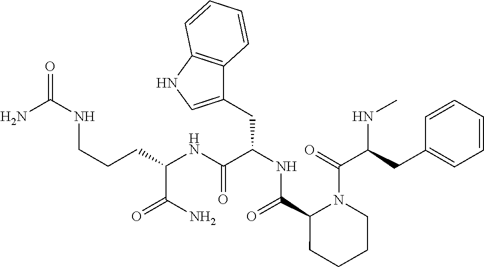 [SMILES: CN[C@@H](Cc1ccccc1)C(=O)N1CCCC[C@H]1C(=O)N[C@@H](Cc1c[nH]c2ccccc12)C(=O)N[C@@H](CCCNC(N)=O)C(N)=O] | 632.34 | >10 μM | 0.62 ± 0.04 |
| 158D11 | 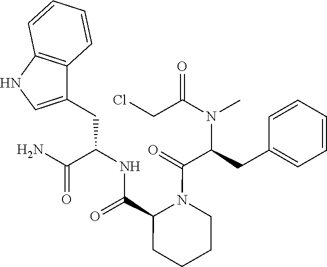 [SMILES: CN(C(=O)CCl)[C@@H](Cc1ccccc1)C(=O)N1CCCC[C@H]1C(=O)N[C@@H](Cc1c[nH]c2ccccc12)C(N)=O] | 551.23 | 71 ± 4 | −10.66 ± 0.04 |
| 158D10 | 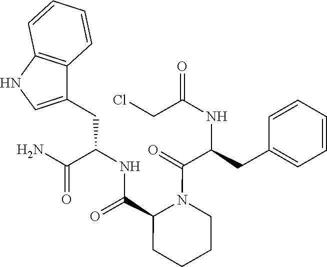 [SMILES: NC(=O)[C@H](Cc1c[nH]c2ccccc12)NC(=O)[C@@H]1CCCCN1C(=O)[C@H](Cc1ccccc1)NC(=O)CCl] | 537.21 | 146 ± 19 | −10.40 ± 0.04 |

Structures of three analogs of BJP-07-017-3 and their characterization by the DELFIA displacement assay and the denaturation thermal shift assay. In DELFIA assay, standard deviation values are obtained from duplicate measurements. In the thermal shift assay, standard errors are reported from four replicates measurements.

As expected, the elimination of the electrophile that reacts with Cys113 in BJP-07-017-3 (compound 158B3) (Table 2) resulted in an agent that is poorly active (IC₅₀>10 μM), and that did not cause a significant denaturation thermal shift (ΔTₘ=+0.62° C.). A minimal core structure of BJP-07-017-3 was then synthesized and tested. As expected, it resulted in an agent that was less active than BJP-07-17-3 in the DELFIA assay (agent 158D11, IC₅₀=71 nM) (FIG. 2). However, surprisingly, and unexpectedly, while 158D11 was less active in binding Pin1 as assessed by the DELFIA assay, it could cause a sizable negative denaturation thermal shift, ΔTₘ=−10.66° C. (Table 2). The elimination of the N-methyl of Phenylalanine residue (agent 158D10) resulted in a higher IC₅₀ value, but no change was observed in the thermal 5 destabilization of the protein. Hence, based on these surprising observations, further iterative rounds of structure activity relationships (SAR) studies were performed around the core structure of 158D10 in the attempt to obtain agents with increased binding affinity for Pin1 and that could also cause Pin1 thermal destabilization. Again, the hypothesis was that such agents would in turn cause Pin1 degradation in cells.

Based on those observations, several structure-activity modifications were applied to 158D10 and iteratively the agents were assessed in both the DELFIA assay and the denaturation thermal shift. First, analogs with derivatives of the phenylalanine and of the tryptophan in 158D10 were synthesized and tested (Table 3).

TABLE 3

| | | | DELFIA IC₅₀ | |
|---|---|---|---|---|
| Name | Structure | MW | (nM) | ΔTm (° C.) |
| 158D9 | 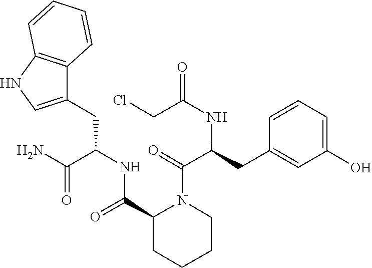 [SMILES: NC(=O)[C@H](Cc1c[nH]c2ccccc12)NC(=O)[C@@H]1CCCCN1C(=O)[C@H](Cc1cccc(O)c1)NC(=O)CCl] | 553.21 | 34 ± 2 | −8.28 ± 0.04 |
| 158E7 | 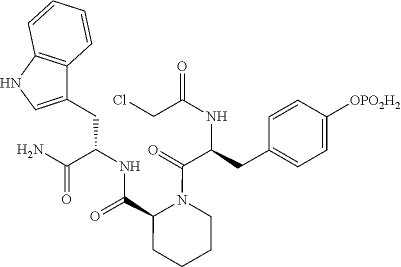 | 633.17 | 327 ± 25 | −7.08 ± 0.22 |
| 158G2 | 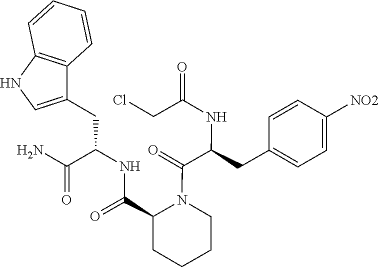 [SMILES: NC(=O)[C@H](Cc1c[nH]c2ccccc12)NC(=O)[C@@H]1CCCCN1C(=O)[C@H](Cc1ccc([N+](=O)[O-])cc1)NC(=O)CCl] | 582.20 | 134 ± 13 | −10.16 ± 0.04 |
| 158G3 | 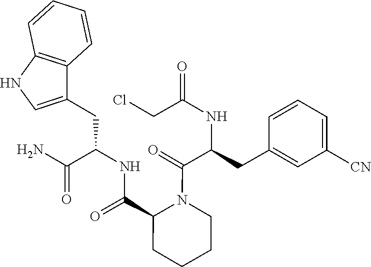 [SMILES: N#Cc1cccc(C[C@H](NC(=O)CCl)C(=O)N2CCCC[C@H]2C(=O)N[C@@H](Cc2c[nH]c3ccccc23)C(N)=O)c1] | 562.21 | 132 ± 21 | −10.06 ± 0.07 |
| 158G4 | 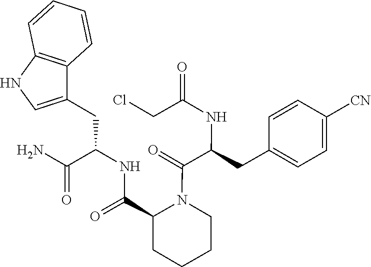 [SMILES: N#Cc1ccc(C[C@H](NC(=O)CCl)C(=O)N2CCCC[C@H]2C(=O)N[C@@H](Cc2c[nH]c3ccccc23)C(N)=O)cc1] | 562.21 | 118 ± 21 | −9.71 ± 0.07 |
| 158G5 | 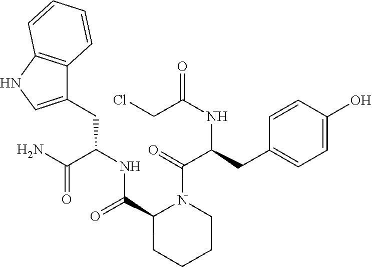 [SMILES: NC(=O)[C@H](Cc1c[nH]c2ccccc12)NC(=O)[C@@H]1CCCCN1C(=O)[C@H](Cc1ccc(O)cc1)NC(=O)CCl] | 553.21 | 119 ± 6 | −9.29 ± 0.04 |
| 158H5 | 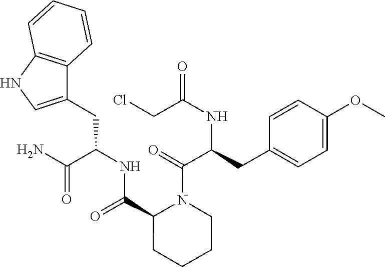 [SMILES: COc1ccc(C[C@H](NC(=O)CCl)C(=O)N2CCCC[C@H]2C(=O)N[C@@H](Cc2c[nH]c3ccccc23)C(N)=O)cc1] | 567.22 | 82.4 ± 0.4 | −9.82 ± 0.08 |
| 158G10 | 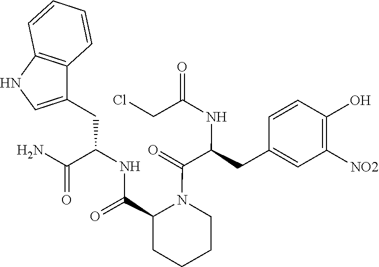 [SMILES: NC(=O)[C@H](Cc1c[nH]c2ccccc12)NC(=O)[C@@H]1CCCCN1C(=O)[C@H](Cc1ccc(O)c([N+](=O)[O-])c1)NC(=O)CCl] | 599.04 | 183 ± 12 | −10.44 ± 0.17 |
| 158G11 | 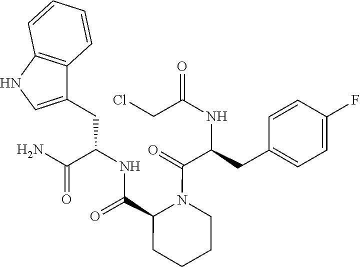 [SMILES: NC(=O)[C@H](Cc1c[nH]c2ccccc12)NC(=O)[C@@H]1CCCCN1C(=O)[C@H](Cc1ccc(F)cc1)NC(=O)CCl] | 556.03 | 146 ± 12 | −10.57 ± 0.11 |
| 158G12 | 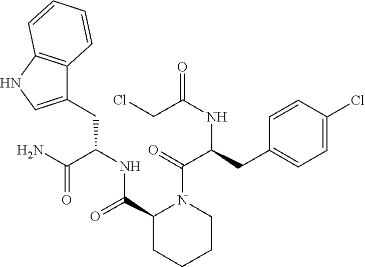 [SMILES: NC(=O)[C@H](Cc1c[nH]c2ccccc12)NC(=O)[C@@H]1CCCCN1C(=O)[C@H](Cc1ccc(Cl)cc1)NC(=O)CCl] | 571.17 | 527 ± 38 | −12.19 ± 0.07 |
| 158H1 | 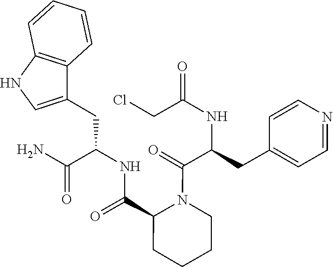 [SMILES: NC(=O)[C@H](Cc1c[nH]c2ccccc12)NC(=O)[C@@H]1CCCCN1C(=O)[C@H](Cc1ccncc1)NC(=O)CCl] | 538.21 | 99 ± 4 | −9.18 ± 0.07 |
| 158F9 | 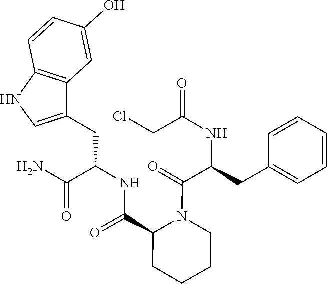 [SMILES: NC(=O)[C@H](Cc1c[nH]c2ccc(O)cc12)NC(=O)[C@@H]1CCCCN1C(=O)[C@H](Cc1ccccc1)NC(=O)CCl] | 553.21 | 135 ± 1 | −9.99 ± 0.00 |
| 158F10 | 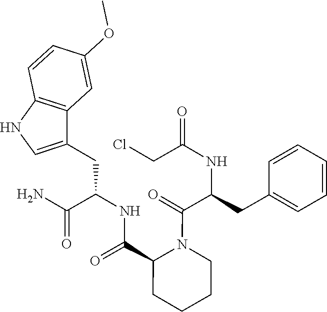 [SMILES: COc1ccc2[nH]cc(C[C@H](NC(=O)[C@@H]3CCCCN3C(=O)[C@H](Cc3ccccc3)NC(=O)CCl)C(N)=O)c2c1] | 567.22 | 126 ± 5 | −10.16 ± 0.07 |
| 158G6 | 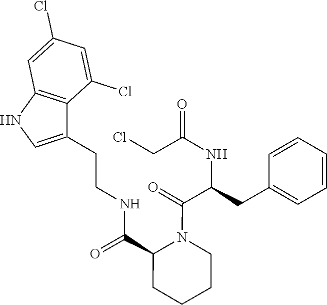 [SMILES: O=C(CCl)N[C@@H](Cc1ccccc1)C(=O)N1CCCC[C@H]1C(=O)NCCc1c[nH]c2cc(Cl)cc(Cl)c12] | 562.13 | 327 ± 18 | +1.04 ± 0.08 (70%) −16.15 ± 0.49 (30%) |
| 164A2 | 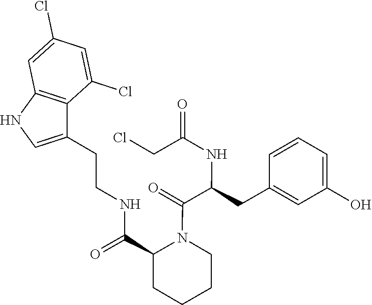 [SMILES: O=C(CCl)N[C@@H](Cc1cccc(O)c1)C(=O)N1CCCC[C@H]1C(=O)NCCc1c[nH]c2cc(Cl)cc(Cl)c12] | 578.12 | 289 ± 23 | −0.25 ± 0.31 (10%) −20.10 ± 0.05 (90%) |
| 158G7 | 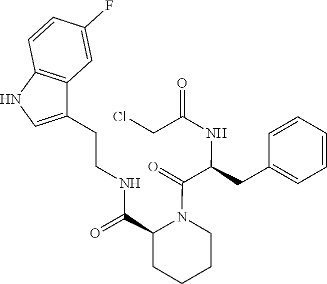 [SMILES: O=C(CCl)N[C@@H](Cc1ccccc1)C(=O)N1CCCC[C@H]1C(=O)NCCc1c[nH]c2ccc(F)cc12] | 512.20 | 1037 ± 316 | −0.45 ± 0.15 (50%) −14.28 ± 0.28 (50%) |
| 158G9 | 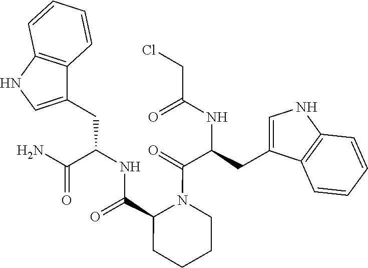 [SMILES: NC(=O)[C@H](Cc1c[nH]c2ccccc12)NC(=O)[C@@H]1CCCCN1C(=O)[C@H](Cc1c[nH]c2ccccc12)NC(=O)CCl] | 577.08 | 44 ± 8 | −9.42 ± 0.18 |

Structure activity relationship studies with analogs of 158D10. Compound characterizations via DELFIA displacement assay and denaturation thermal shift assay are reported. In the analysis by thermal shift of Pin1 in the presence of certain agents, two peaks were observed, likely resulting from incomplete complex formation under the experimental conditions. In DELFIA assay, standard deviation values are obtained from duplicate measurements. In the thermal shift assay, standard errors are reported from four replicates measurements.

Notably, replacement of the Phe with a Trp residue led to agent 158G9 that resulted in an IC₅₀ value of 43 nM. Hence, several modifications were subsequently introduced at the side chains of both Trp residues present in 158G9, and at the homo-Proline. These SAR data are summarized in Table 4.

TABLE 4

| | | | DELFIA | |
|---|---|---|---|---|
| Name | Structure | MW | IC₅₀ (nM) | ΔTm (° C.) |
| 158H7 | 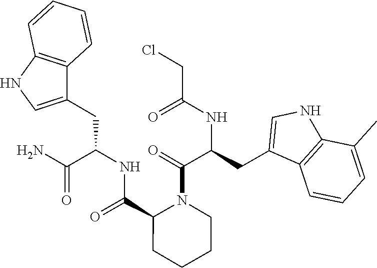 | 590.24 | 91 ± 9 | −10.11 ± 0.20 |
| 158H3 | 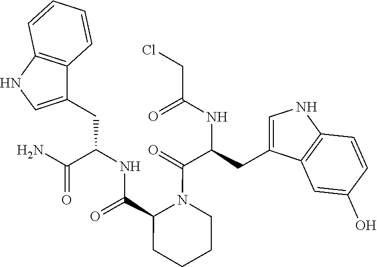 [SMILES: NC(=O)[C@H](Cc1c[nH]c2ccccc12)NC(=O)[C@@H]1CCCCN1C(=O)[C@H](Cc1c[nH]c2ccc(O)cc12)NC(=O)CCl] | 592.22 | 56 ± 5 | −6.56 ± 0.04 |
| 158H8 | 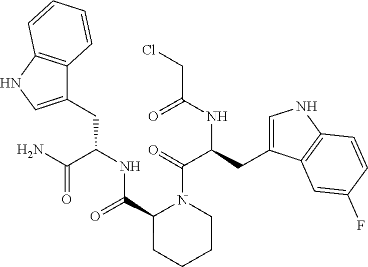 [SMILES: NC(=O)[C@H](Cc1c[nH]c2ccccc12)NC(=O)[C@@H]1CCCCN1C(=O)[C@H](Cc1c[nH]c2ccc(F)cc12)NC(=O)CCl] | 694.21 | 43.5 ± 0.8 | −9.81 ± 0.16 |
| 158H9 | 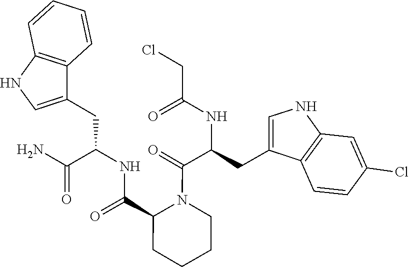 [SMILES: NC(=O)[C@H](Cc1c[nH]c2ccccc12)NC(=O)[C@@H]1CCCCN1C(=O)[C@H](Cc1c[nH]c2cc(Cl)ccc12)NC(=O)CCl] | 610.19 | 21.5 ± 0.6 | −8.41 ± 0.04 |
| 158H6 | 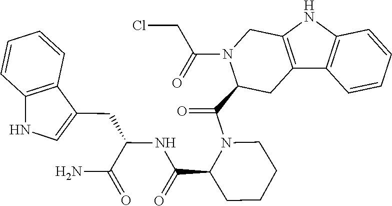 [SMILES: NC(=O)[C@H](Cc1c[nH]c2ccccc12)NC(=O)[C@@H]1CCCCN1C(=O)[C@@H]1Cc2c([nH]c3ccccc23)CN1C(=O)CCl] | 588.22 | 6.8 ± 0.1 | −9.85 ± 0.05 |
| 158H10 | 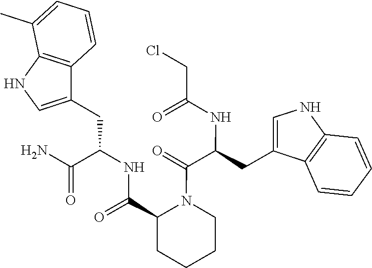 [SMILES: Cc1cccc2c(C[C@H](NC(=O)[C@@H]3CCCCN3C(=O)[C@H](Cc3c[nH]c4ccccc34)NC(=O)CCl)C(N)=O)c[nH]c12] | 590.24 | 15 ± 1 | −7.38 ± 0.11 |
| 158H11 | 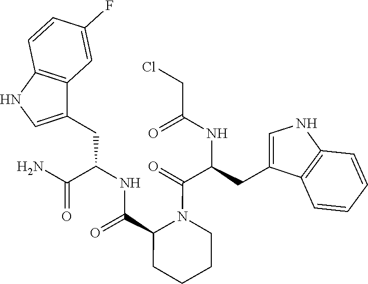 [SMILES: NC(=O)[C@H](Cc1c[nH]c2ccc(F)cc12)NC(=O)[C@@H]1CCCCN1C(=O)[C@H](Cc1c[nH]c2ccccc12)NC(=O)CCl] | 594.22 | 14.4 ± 0.1 | −7.55 ± 0.05 |
| 158H12 | 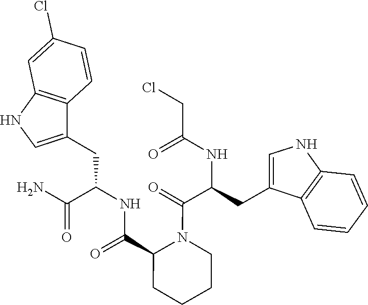 [SMILES: NC(=O)[C@H](Cc1c[nH]c2cc(Cl)ccc12)NC(=O)[C@@H]1CCCCN1C(=O)[C@H](Cc1c[nH]c2ccccc12)NC(=O)CCl] | 610.19 | 31 ± 1 | −9.21 ± 0.08 |
| 164A3 | 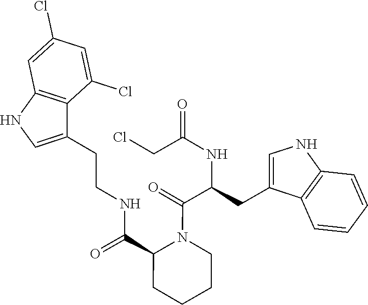 [SMILES: O=C(CCl)N[C@@H](Cc1c[nH]c2ccccc12)C(=O)N1CCCC[C@H]1C(=O)NCCc1c[nH]c2cc(Cl)cc(Cl)c12] | 601.14 | 1175 ± 45 | −13.68 ± 0.20 |
| 164A1 | 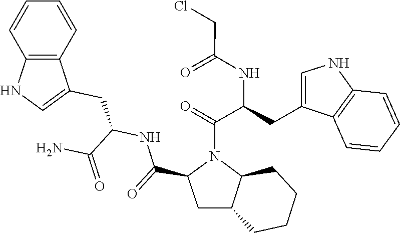 [SMILES: NC(=O)[C@H](Cc1c[nH]c2ccccc12)NC(=O)[C@@H]1C[C@H]2CCCC[C@@H]2N1C(=O)[C@H](Cc1c[nH]c2ccccc12)NC(=O)CCl] | 616.26 | 1826 ± 394 | −0.38 ± 0.15 (80%) −23.86 ± 0.35 (20%) |
| 164A4 | 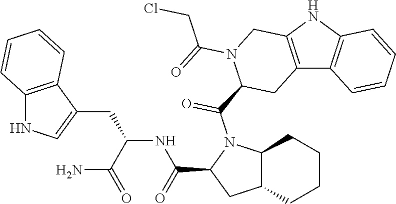 [SMILES: NC(=O)[C@H](Cc1c[nH]c2ccccc12)NC(=O)[C@@H]1C[C@H]2CCCC[C@@H]2N1C(=O)[C@@H]1Cc2c([nH]c3ccccc23)CN1C(=O)CCl] | 628.26 | 35 ± 5 | −20.46 ± 0.04 |
| 164A5 | 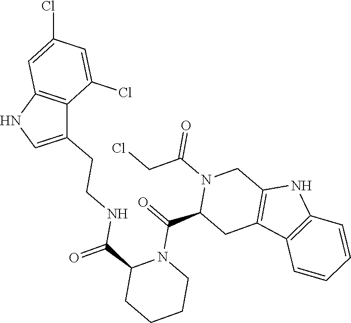 [SMILES: O=C(NCCc1c[nH]c2cc(Cl)cc(Cl)c12)[C@@H]1CCCCN1C(=O)[C@@H]1Cc2c([nH]c3ccccc23)CN1C(=O)CCl] | 613.14 | 433 ± 65 | +0.66 ± 0.28 (70%) −17.6 ± 0.39 (30%) |
| 164A7 | 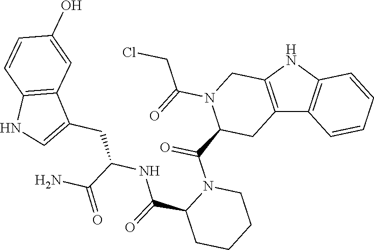 [SMILES: NC(=O)[C@H](Cc1c[nH]c2ccc(O)cc12)NC(=O)[C@@H]1CCCCN1C(=O)[C@@H]1Cc2c([nH]c3ccccc23)CN1C(=O)CCl] | 604.22 | 5.0 ± 0.5 | −7.85 ± 0.16 |
| 164A8 | 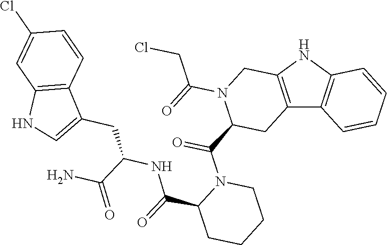 [SMILES: NC(=O)[C@H](Cc1c[nH]c2cc(Cl)ccc12)NC(=O)[C@@H]1CCCCN1C(=O)[C@@H]1Cc2c([nH]c3ccccc23)CN1C(=O)CCl] | 622.19 | 7.5 ± 0.1 | −10.71 ± 0.05 |
| 164A9 | 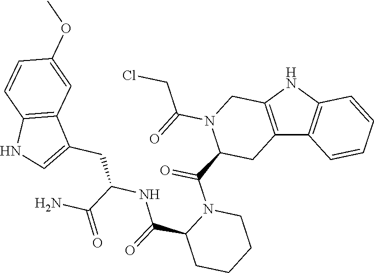 [SMILES: COc1ccc2[nH]cc(C[C@H](NC(=O)[C@@H]3CCCCN3C(=O)[C@@H]3Cc4c([nH]c5ccccc45)CN3C(=O)CCl)C(N)=O)c2c1] | 618.24 | 2.8 ± 0.1 | −9.00 ± 0.05 |
| 164A10 | 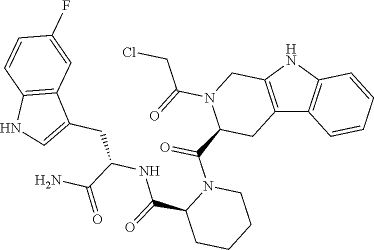 [SMILES: NC(=O)[C@H](Cc1c[nH]c2ccc(F)cc12)NC(=O)[C@@H]1CCCCN1C(=O)[C@@H]1Cc2c([nH]c3ccccc23)CN1C(=O)CCl] | 606.22 | 4.1 ± 0.2 | −8.95 ± 0.04 |
| 164A11 | 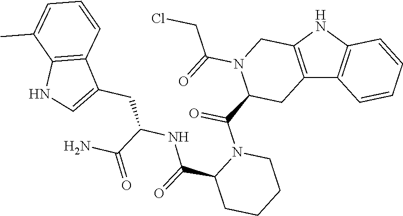 [SMILES: Cc1cccc2c(C[C@H](NC(=O)[C@@H]3CCCCN3C(=O)[C@@H]3Cc4c([nH]c5ccccc45)CN3C(=O)CCl)C(N)=O)c[nH]c12] | 602.24 | 7.0 ± 0.6 | +10.45 ± 0.07 |
| 164A12 | 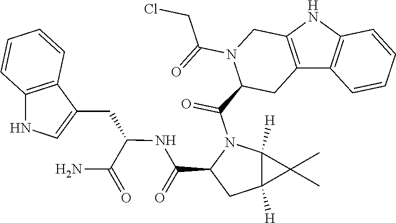 [SMILES: CC1(C)[C@H]2C[C@@H](C(=O)N[C@@H](Cc3c[nH]c4ccccc34)C(N)=O)N(C(=O)[C@@H]3Cc4c([nH]c5ccccc45)CN3C(=O)CCl)[C@H]21] | 614.24 | 44 ± 1 | +1.03 ± 0.13 (30%) −19.18 ± 0.07 (70%) |

Structure activity relationships data relative analogs of 158G9. Characterizations by DELFIA displacement assay and denaturation thermal shift assay are reported. For some compounds two denaturation transitions were observed as indicated, likely resulting from incomplete complex formation under the experimental conditions. In DELFIA assay, standard deviation values are obtained from duplicate measurements. In the thermal shift assay, standard errors are reported from four replicates measurements.

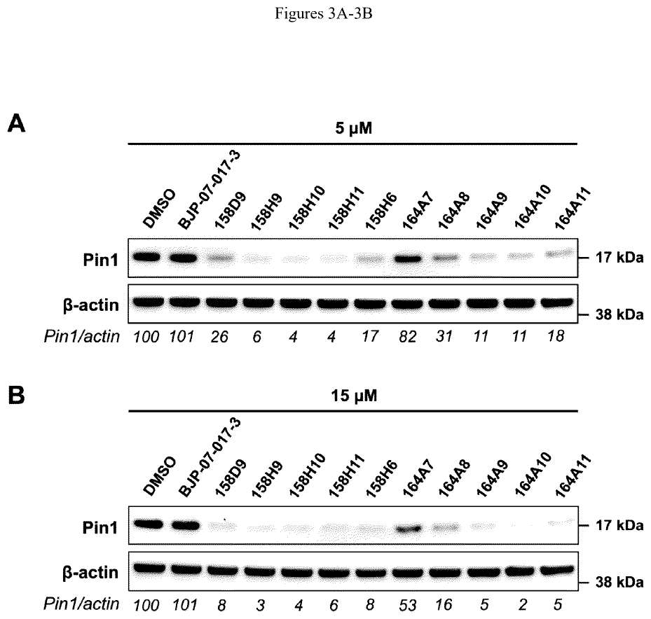

These data resulted in the selection of 10 agents that presented the lowest IC₅₀ values for Pin1 (IC₅₀<34 nM) and that could induce sizable negative ΔTₘ values (ΔTₘ<−8° C.). These agents were subsequently tested for protein degradation in cell against the pancreatic cancer cell line BxPC3 (FIG. 3).

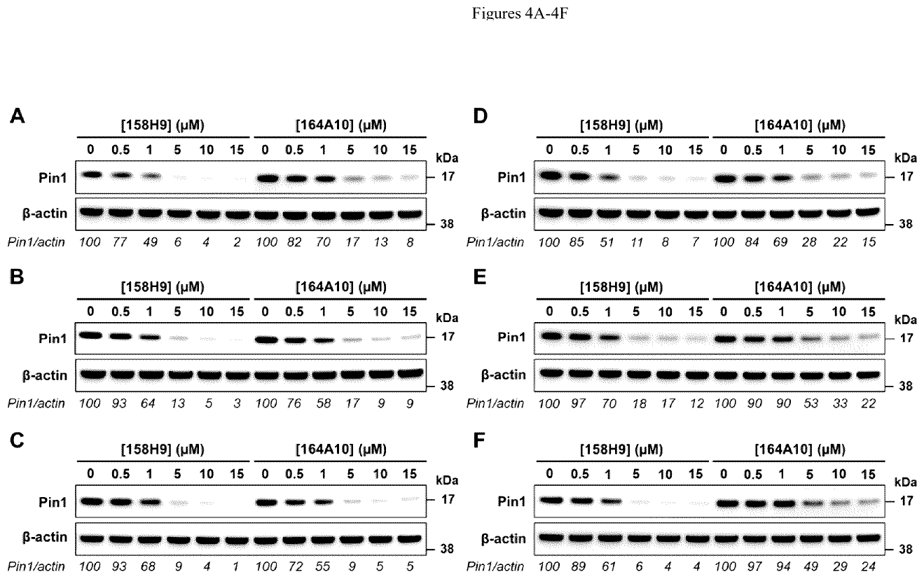

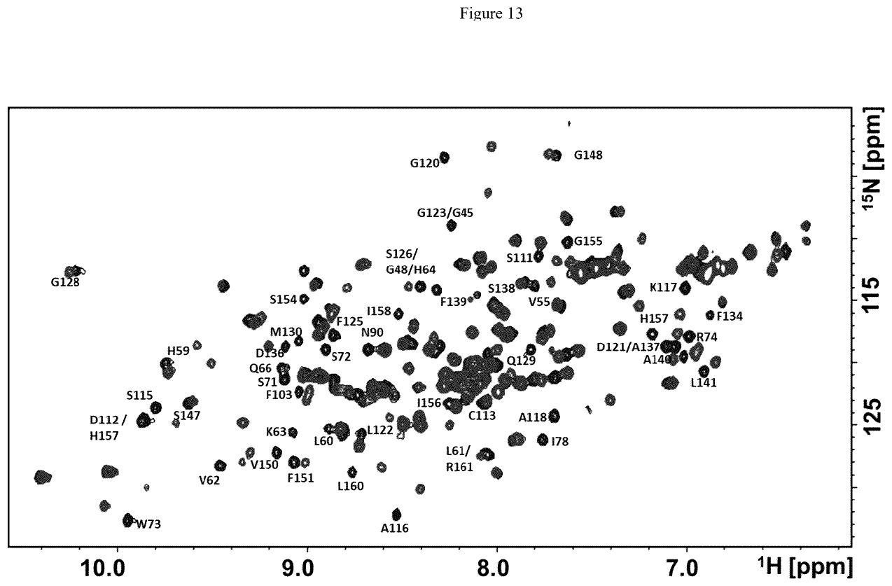

Compounds with high affinity for Pin1 and that caused Pin1 thermal destabilization in vitro (ΔTₘ<−8° C.) also resulted in increased Pin1 protein degradation in cell (FIG. 3). On the contrary, and in agreement with the central hypothesis, agents that did not cause thermal denaturation also failed in inducing Pin1 degradation cell (FIG. 12). Of the agents that bound potently to Pin1 and that also increased Pin1 instability to thermal denaturation in vitro, compounds 158H₉ and 164A10 were selected for further evaluations against a panel of human cancer cell lines (FIG. 4). In these studies, it was evident that both agents were very effective in degrading Pin1 in all tested human cancer cell lines with DC₅₀ values (the concentration at which 50% degradation was observed)˜500 nM and Dₘₐₓ (the maximal level of degradation) close to 100% with all cell lines when exposed to 5 μM of either agent (FIG. 4).

### Destabilized Pin1 Gets Degraded Via the Ubiquitin-Proteasome System.

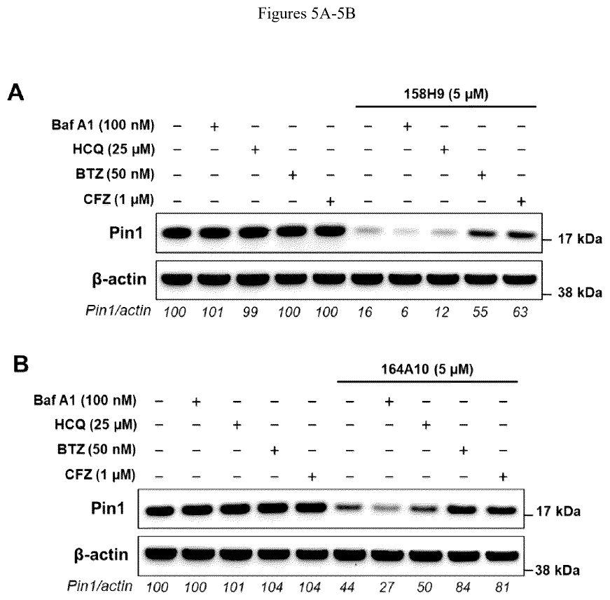

To decipher, at the molecular level, the mechanisms that may correlate compound induced thermal instability in vitro and with compound induced protein degradation in cell, additional experiments were conducted. First, it was found that Pin1 degradation induced by the agents was likely due to the ubiquitin-proteosome system, based on the observation that Pin1 degradation was reversed by combination treatment of cell lines with the proteasome inhibitors carfilzomib (CFZ) or bortezomib (BTZ), while autophagy inhibitors hydroxychloroquine (HCQ) or bafilomycin A1 (Baf A1) were ineffective (FIG. 5). Previous studies had identified Pin1 residue Lys 117 as a site of ubiquitination and proteasomal degradation (Lepore A, et al. (2021) Hepatology 74(5):2561-2579). Indeed, a Pin1 Lys117Arg mutant presented an increased half-life in cells, likely resulting from the absence of Pin1 mono-ubiquitination at Lys 117 (Lepore A, et al. (2021) Hepatology 74(5):2561-2579).

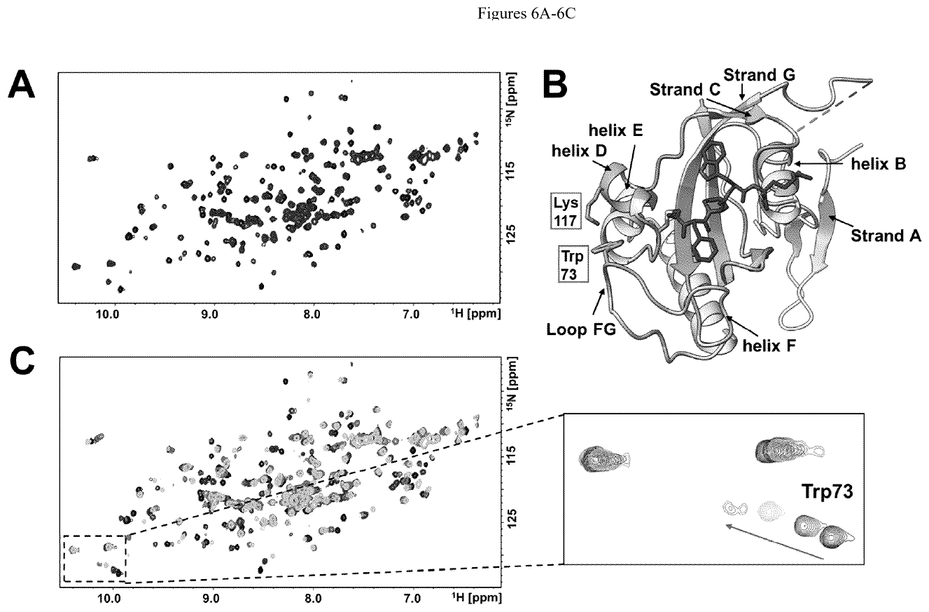

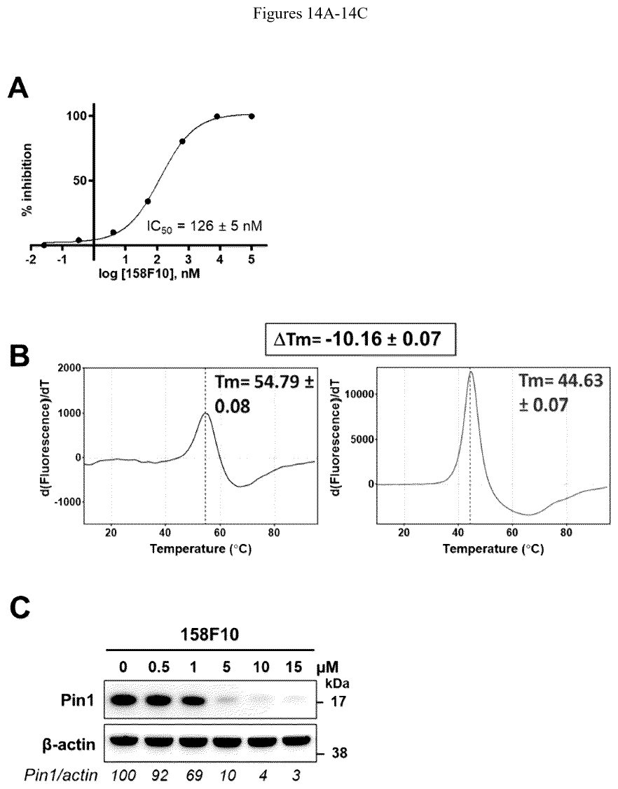

Subsequently, ligand binding studies were conducted using solution NMR spectroscopy measurement with a ¹⁵N-labeled sample of Pin1. Intriguingly, analysis of backbone ¹⁵N, ¹H chemical shift perturbations upon binding of selected agents revealed that these compounds, such as agent 158F10 and agent 164A9 reported in FIG. 6A, C, affected not only residues located within the binding site, but also in helix D (FIG. 6B and FIG. 13), where residue Lys117 is located. This is also evident by monitoring the side chain ¹H^{ε}/¹⁵N^{ε} resonances of Trp 73, engaged in a cation-π interaction with Lys 117 in the apo protein (FIG. 6). It was found that Trp 73 side chain resonances are either largely perturbed or broaden beyond detection by agents that cause Pin1 degradation compared to other agents that are less effective in causing degradation such as sulfopin for example (FIG. 6). These observations suggested that perhaps helix D and the position of Trp 73 upon binding can more easily expose Lys117 to ubiquitination and degradation.

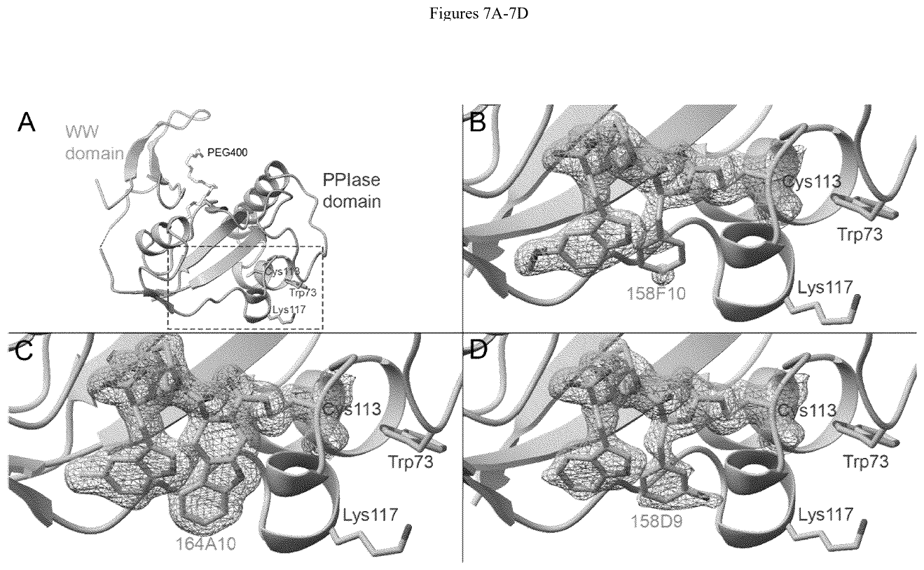

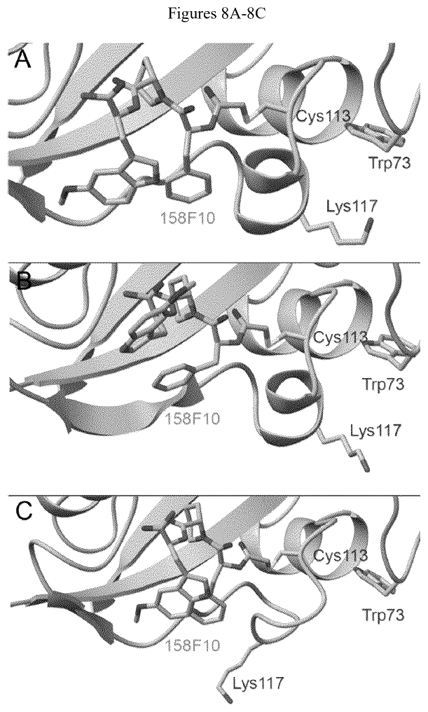

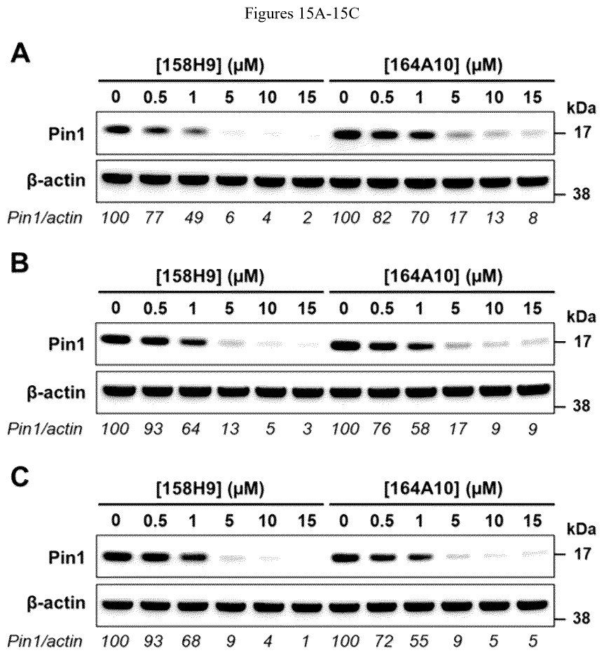

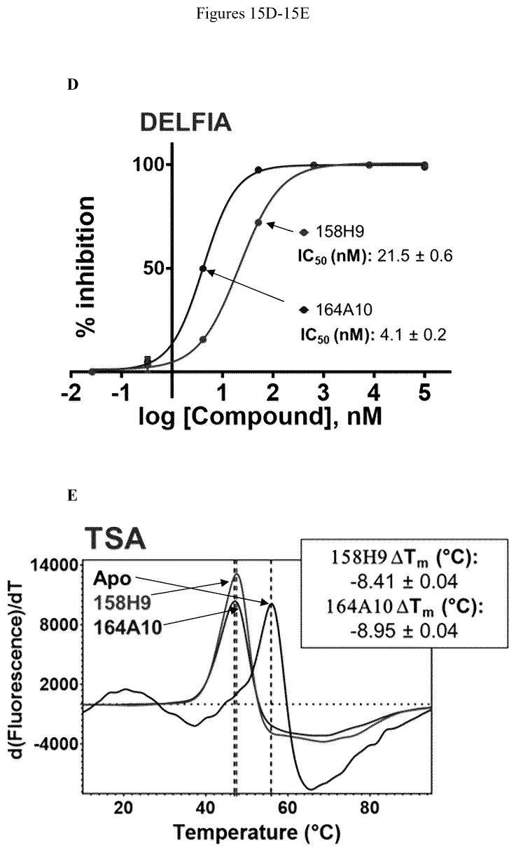

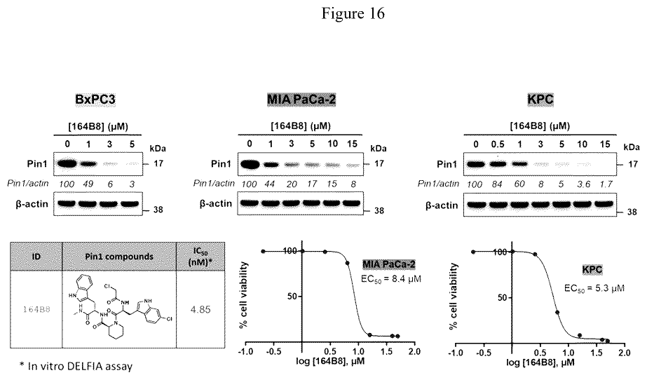

To further test those hypotheses, the X-ray structure of selected agents in complex with Pin1 was solved. When compounds 164A10 (FIG. 7C), 158D9 (FIG. 7D), or 158F10 (FIG. 7B, FIG. 14) were soaked in the crystals of Pin1, contiguous electron density was observed between the compound and the protein at Cys113, and the position of Lys117 and Trp 73 appeared similar to what observed in the apo form of the protein (FIG. 7A). However, when ligand 158F10 was co-crystallized with Pin1, it assumed slightly different conformations, including unfolding of the helix D and exposure of Lys117 (FIG. 8).

Taken together these studies identify 158H₉ and 164A10 and related compounds as the most potent targeted Pin1 agents reported to date, and that have been rationally designed to cause Pin1 degradation in cancer cells. These agents can be used to further probe the role of Pin1 in cancer and could provide invaluable steppingstones in the design of Pin1 based therapeutics.

### Discussion and Conclusions

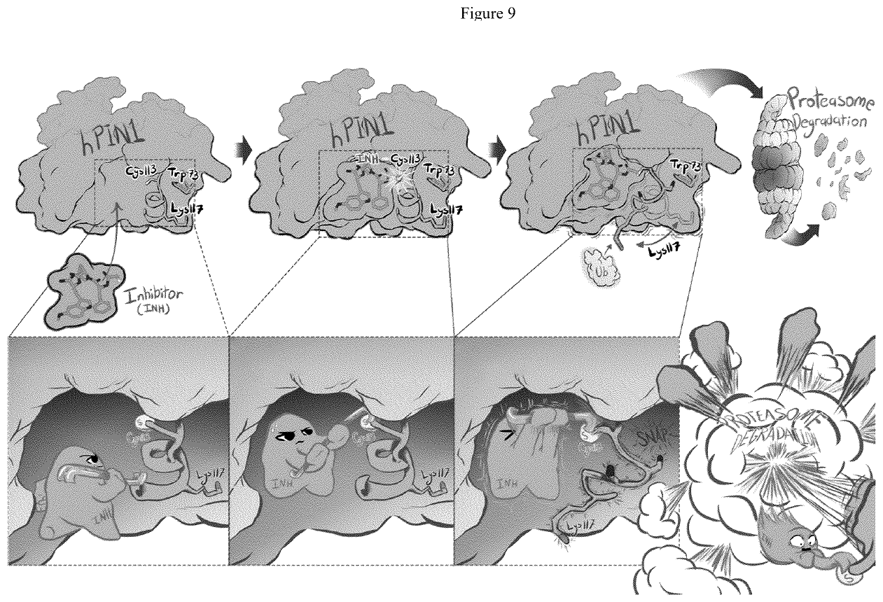

In recent years, there have been tremendous advances in drug discovery research for challenging drug targets. For example, there has been a resurgence of rationally designed covalent drugs, as those present favorable pharmacodynamics and pharmacokinetics properties over non-covalent ligands (Akher F B, Farrokhzadeh A, & Soliman M E S (2019) Chem Biodivers 16(3):e1800518; Singh J, Petter R C, Baillie T A, & Whitty A (2011) Nat Rev Drug Discov 10(4):307-317; Kalgutkar A S & Dalvie D K (2012) Expert Opin Drug Discov 7(7):561-581; Mah R, Thomas J R, & Shafer C M (2014) Bioorg Med Chem Lett 24(1):33-39; Baillie T A (2016) Angew Chem Int Ed Engl 55(43):13408-13421; Basu D, Richters A, & Rauh D (2015) Bioorg Med Chem 23(12):2767-2780; Bauer R A (2015) Drug Discov Today 20(9):1061-1073; Engel J, et al. (2015) J Med Chem 58(17):6844-6863; Ghosh A K, Samanta I, Mondal A, & Liu W R (2019) ChemMedChem 14(9):889-906; Lonsdale R & Ward R A (2018) Chem Soc Rev 47(11):3816-3830; Vasudevan A, et al. (2019) Prog Med Chem 58:1-62; Nussinov R & Tsai C J (2015) Annu Rev Pharmacol Toxicol 55:249-267; Adeniyi A A, Muthusamy R, & Soliman M E (2016) Expert Opin Drug Discov 11(1):79-90; Bjij I, et al. (2018) Curr Top Med Chem 18(13):1135-1145; Chaikuad A, Koch P, Laufer S A, & Knapp S (2018) Angew Chem Int Ed Engl 57(16):4372-4385; Wu S, et al. (2016) Biochem Biophys Res Commun 478(3):1268-1273; and Liu Q, et al. (2013) Chem Biol 20(2):146-159). More recently, the field has been focusing on the design of heterobifunctional molecules that bring an E3 ubiquitin ligase in proximity of the drug target. Those approaches, such as proteolysis targeting chimera (PROTAC) and molecular glues (Bekes M, Langley D R, & Crews C M (2022) Nat Rev Drug Discov 21(3):181-200; Hu Z & Crews C M (2022) Chembiochem 23(2):e202100270; Paiva S L & Crews C M (2019) Curr Opin Chem Biol 50:111-119; and Ramachandran S & Ciulli A (2021) Curr Opin Struct Biol 67:110-119), are based on hijacking the ubiquitin-proteasome system to induce the ubiquitination and subsequent proteasomal degradation of the drug target in cell. The advantage of the PROTAC approach is that the bi-functional molecule can be composed to any protein target binder, hence not necessarily a molecule that inhibits protein function, thus largely expanding the toolkit for the design of possible therapeutics. Here, the studies are based on the hypothesis that agents that can bind potently (e.g., covalently) to a given target and that can also cause its thermal instability in vitro, may also cause target degradation in cell. Hence, when this hypothesis was applied to the design of Pin1 degraders, not only a biochemical displacement assay was used to rank order agents during structure activity relationships studies, but also denaturation thermal shift measurements, iteratively selecting for agents that cause protein thermal destabilization. In fact, compounds that were potent but did not cause thermal instability also resulted in less effective Pin1 degrading agents in cell (FIG. 1, FIG. 12). A possible molecular mechanism, albeit still speculative, could be stipulated whereby compound binding causes conformational instability in region of the protein that encompass Lys 117, that in cell is then targeted by the ubiquitin-proteasome system for degradation (FIG. 9). Therefore, it appears that the agents may function as molecular crowbars, that open-up the protein and induce conformational instabilities that signal the protein for degradation in cell (FIG. 9).

Hence, a series of rationally designed potent Pin1 degraders that do not require the synthesis of PROTACs or other molecular glues to bring the target in proximity to the ubiquitin proteasome degradation machinery have been identified. While the destabilizing ligands cause the degradation of Pin1 via the ubiquitin-proteasome system, it can be envisioned that in other protein targets the ligand induced unfolding may activate other protein recycling mechanisms, such the lysosomal degradation, for example. Hence, the studies propose possibly a new design strategy, aimed at obtaining protein binders that induce target instability in vitro and target degradation in cell. These agents can be termed, molecular crowbars (FIG. 9), and their design strategy can represent a new and efficient way to induce protein degradation without needing the synthesis of chimeric bi-dentate agents like in PROTAC or molecular glues.

In conclusion, a potent compound series with ligands that are effective in causing Pin1 degradation in several human cancer cell lines have been identified. These open the way to a variety of phenotypic assays and target validation studies in cellular and animal models of several cancer types to ascertain if those Pin1 inhibitors can be translated into effective anti-cancer therapies.

### Materials and Methods

The reagents and solvents used in the experiment were commercially sourced, including most of the Fmoc-protected amino acids and resins for solid-phase synthesis. To determine the concentration of stock solutions, NMR spectra in d6-DMSO were recorded on a Bruker Avance III 700 MHz with a TCI cryoprobe. For quality control and to ensure the solubility of the compounds used in all studies, ¹H NMR measurements were conducted in the assay buffer. High-resolution mass spectral data were obtained using an Agilent 6545 Q-TOF LC/MS instrument (Table S1), and RP-HPLC purifications were carried out on a JASCO preparative system equipped with a PDA detector and a fraction collector controlled by a ChromNAV system (JASCO) on a XTerra C18 10 μm 10×250 mm² (Waters). The purity of the tested compounds was determined by HPLC, and all compounds have a purity of ≥95%.

TABLE S1
Mass-spectrometry data of the compounds. All the compounds
were analyzed using an Agilent 6545 QTOF LC/MS instrument.

| ID | Calcd [M] | Obs. [M + H]⁺ (m/z) |
|---|---|---|
| 158B3 | 632.3445 | 633.3520 |
| 158D9 | 553.2090 | [M + Na]⁺ = 576.1984 |
| 158D10 | 537.2146 | [M + Na]⁺ = 560.2041 |
| 158D11 | 551.2299 | [M + Na]⁺ = 574.2191 |
| 158E7 | 633.1762 | 634.1839 |
| 158F9 | 553.2102 | 554.2148 |
| 158F10 | 567.2260 | 568.2319 |
| 158G2 | 582.1997 | 583.2049 |
| 158G3 | 562.2098 | 563.2168 |
| 158G4 | 562.2095 | 563.2153 |
| 158G5 | 553.2092 | [M + Na]⁺ = 576.1990 |
| 158G6 | 562.1305 | 565.1558 |
| 158G7 | 512.2000 | [M + Na]⁺ = 535.1894 |
| 158G9 | 576.2263 | [M + Na]⁺ = 599.2157 |
| 158G10 | 598.1943 | 599.2125 |
| 158G11 | 555.2068 | [M + Na]⁺ = 578.1962 |
| 158G12 | 571.1740 | [M + Na]⁺ = 594.1634 |
| 158H1 | 538.2108 | 539.2184 |
| 158H3 | 592.2195 | [M + Na]⁺ = 615.2089 |
| 158H5 | 567.2238 | [M + Na]⁺ = 590.2133 |
| 158H6 | 588.2245 | [M + Na]⁺ = 611.2139 |
| 158H7 | 590.2398 | [M + Na]⁺ = 613.2293 |
| 158H8 | 594.2153 | [M + Na]⁺ = 617.2047 |
| 158H9 | 610.1863 | [M + Na]⁺ = 633.1757 |
| 158H10 | 590.2406 | [M + Na]⁺ = 613.2301 |
| 158H11 | 594.2159 | [M + Na]⁺ = 617.2053 |
| 158H12 | 610.1861 | [M + Na]⁺ = 633.1755 |
| 164A1 | 616.2566 | 617.2641 |
| 164A2 | 578.1254 | [M + Na]⁺ = 503.1471 |
| 164A3 | 601.1413 | [M + Na]⁺ = 624.1304 |
| 164A4 | 628.2548 | 629.2617 |
| 164A5 | 613.1414 | 616.1643 |
| 164A7 | 604.2205 | [M + Na]⁺ = 627.2099 |
| 164A8 | 622.1862 | [M + Na]⁺ = 645.1758 |
| 164A9 | 618.2357 | [M + Na]⁺ = 641.2251 |
| 164A10 | 606.2158 | [M + Na]⁺ = 629.2052 |
| 164A11 | 602.2408 | [M + Na]⁺ = 625.2315 |
| 164A12 | 614.2408 | 615.2473 |

All agents were synthesized in-house by standard solid-phase Fmoc peptide synthesis protocols on Rink Amide resin or on BAL resin, and the final reaction with 2-chloroacetyl chloride was performed in solution. To perform each coupling reaction, 3 equivalents of Fmoc-AA, 3 equivalents of HATU, 3 equivalents of Oxima Pure, and 5 equivalents of DIPEA were mixed in 1 mL of DMF. The reaction was allowed to proceed for 1 hour. To remove the Fmoc protecting group, the resin-bound peptide was treated twice with 20% piperidine in DMF for 5 minutes and 20 minutes, respectively. The loading of the amine on the BAL resin was performed by adding 3 equivalents of amine (in DMF) to the solid support and shaking for 30 minutes before the addition of NaBH(OAc)₃ (room temperature, overnight). Once the peptide chain was complete, it was cleaved from the solid support with a cleavage solution containing TFA/TIS/water (94:3:3) for 3 h. The cleaving solution was then filtered from the resin and evaporated under reduced pressure. The amino acids used in each synthesis are reported in Table S2

TABLE S2
Summary of amino acids used to synthesize each compound (P1 = N
terminus, P2 = central position, P3 = C terminus).

| ID | P1 | P2 | P3 |
|---|---|---|---|
| 158D9 | Fmoc-Phe(3-OH)-OH | Fmoc-Pip-OH | Fmoc-L-Trp-OH |
| 158D10 | Fmoc-L-Phe-OH | Fmoc-Pip-OH | Fmoc-L-Trp-OH |
| 158D11 | Fmoc-N-Methyl-L-Phe- | Fmoc-Pip-OH | Fmoc-L-Trp-OH |
| | OH | | |
| 158E7 | Fmoc-Phe(4-OPO₃H₂)- | Fmoc-Pip-OH | Fmoc-L-Trp-OH |
| | OH | | |
| 158F9 | Fmoc-L-Phe-OH | Fmoc-Pip-OH | Fmoc-Trp(5-OH)-OH |
| 158F10 | Fmoc-L-Phe-OH | Fmoc-Pip-OH | Fmoc-Trp(5-MeO)-OH |
| 158G2 | Fmoc-Phe(4-NO₂)-OH | Fmoc-Pip-OH | Fmoc-L-Trp-OH |
| 158G3 | Fmoc-Phe(3-CN)-OH | Fmoc-Pip-OH | Fmoc-L-Trp-OH |
| 158G4 | Fmoc-Phe(4-CN)-OH | Fmoc-Pip-OH | Fmoc-L-Trp-OH |
| 158G5 | Fmoc-Phe(4-OH)-OH | Fmoc-Pip-OH | Fmoc-L-Trp-OH |
| 158G6 | Fmoc-L-Phe-OH | Fmoc-Pip-OH | Fmoc-4,6-diCl- |
| | | | Tryptamine |
| 158G7 | Fmoc-L-Phe-OH | Fmoc-Pip-OH | Fmoc-5-F-Tryptamine |
| 158G9 | Fmoc-L-Trp-OH | Fmoc-Pip-OH | Fmoc-L-Trp-OH |
| 158G10 | Fmoc-Phe(4-OH)-(3- | Fmoc-Pip-OH | Fmoc-L-Trp-OH |
| | NO₂)-OH | | |
| 158G11 | Fmoc-Phe(4-F)-OH | Fmoc-Pip-OH | Fmoc-L-Trp-OH |
| 158G12 | Fmoc-Phe(4-Cl)-OH | Fmoc-Pip-OH | Fmoc-L-Trp-OH |
| 158H1 | Fmoc-4-pyridyl-Ala-OH | Fmoc-Pip-OH | Fmoc-L-Trp-OH |
| 158H3 | Fmoc-Trp(5-OH)-OH | Fmoc-Pip-OH | Fmoc-L-Trp-OH |
| 158H5 | Fmoc-Phe(4-MeO)-OH | Fmoc-Pip-OH | Fmoc-L-Trp-OH |
| 158H6 | Fmoc-Tpi-OH | Fmoc-Pip-OH | Fmoc-L-Trp-OH |
| 158H7 | Fmoc-Trp(7-Me)-OH | Fmoc-Pip-OH | Fmoc-L-Trp-OH |
| 158H8 | Fmoc-Trp(5-F)-OH | Fmoc-Pip-OH | Fmoc-L-Trp-OH |
| 158H9 | Fmoc-Trp(6-Cl)-OH | Fmoc-Pip-OH | Fmoc-L-Trp-OH |
| 158H10 | Fmoc-L-Trp-OH | Fmoc-Pip-OH | Fmoc-Trp(7-Me)-OH |
| 158H11 | Fmoc-L-Trp-OH | Fmoc-Pip-OH | Fmoc-Trp(5-F)-OH |
| 158H12 | Fmoc-L-Trp-OH | Fmoc-Pip-OH | Fmoc-Trp(6-Cl)-OH |
| 164A1 | Fmoc-L-Trp-OH | Fmoc-L- | Fmoc-L-Trp-OH |
| | | octahydroindole- | |
| | | 2-carboxylic acid | |
| 164A2 | Fmoc-Phe(3-OH)-OH | Fmoc-Pip-OH | Fmoc-4,6-diCl-Tryptamine |
| 164A3 | Fmoc-L-Trp-OH | Fmoc-Pip-OH | Fmoc-4,6-diCl- |
| | | | Tryptamine |
| 164A4 | Fmoc-Tpi-OH | Fmoc-L- | Fmoc-L-Trp-OH |
| | | octahydroindole- | |
| | | 2-carboxylic acid | |
| 164A5 | Fmoc-Tpi-OH | Fmoc-Pip-OH | Fmoc-4,6-diCl- Tryptamine |
| 164A7 | Fmoc-Tpi-OH | Fmoc-Pip-OH | Fmoc-Trp(5-OH)-OH |
| 164A8 | Fmoc-Tpi-OH | Fmoc-Pip-OH | Fmoc-Trp(6-C1)-OH |
| 164A9 | Fmoc-Tpi-OH | Fmoc-Pip-OH | Fmoc-Trp(5-MeO)-OH |
| 164A10 | Fmoc-Tpi-OH | Fmoc-Pip-OH | Fmoc-Trp(5-F)-OH |
| 164A11 | Fmoc-Tpi-OH | Fmoc-Pip-OH | Fmoc-Trp(7-Me)-OH |
| 164A12 | Fmoc-Tpi-OH | N-Fmoc- | Fmoc-L-Trp-OH |
| | | (1R,2S,5S)-6,6- | |
| | | dimethy1-3- | |
| | | azabicyclo[3.1.0]h | |
| | | exane- | |
| | | 2-carboxylic acid | |

Reaction with 2-chloroacetylchloride

The purified peptide (1 eq.) was dissolved in DMF and cooled to 0° C. Subsequently, 2-chloroacetyl chloride (3 eq.) and DIPEA (3 eq.) were added dropwise at 0° C. and stirred for 2 h. Afterward, the reaction mixture was allowed to reach room temperature and stirred for 16 h (overnight, room temperature). The purification was performed by RP-HPLC (linear gradient 5→100% ACN/H₂O+0.1% TFA in 30 min).

### Protein Expression and Purification

The cDNA fragment encoding human Pin1 (residues 1-163) was cloned into a pET28a (+) vector with an N-terminal His tag. This vector was then transformed into BL21 (DE3) gold pLysS competent cells and grown in LB medium at 37° C. with 50 μg/mL of Kanamycin until the OD value reached 0.6-0.7. The cells were then induced with 1 mM IPTG overnight at 25° C. After induction, the bacteria were collected by centrifugation and lysed by sonication. The overexpressed protein containing an N-terminal His tag was purified using immobilized metal ion affinity chromatography (IMAC) with a linear gradient of imidazole. The elution buffer used was 25 mM Tris at pH 7.5, 500 mM NaCl, and 500 mM imidazole. Following IMAC purification, the protein was further purified, and buffer exchanged through a size exclusion chromatography with a HiLoad 26/60 Superdex 75 preparative-grade column into an aqueous buffer composed of 50 mM phosphate at pH 7.5, 150 mM NaCl, and 1 mM DTT.

### Nuclear Magnetic Resonance Spectroscopy

NMR spectra were obtained using a Bruker Avance III 700 MHz spectrometer equipped with a TCI cryoprobe. The acquired data were then processed and analyzed with TOPSPIN 4.1.0 (Bruker, Billerica, MA). For each compound, 1D ¹H experiments were acquired, to check concentration and solubility and in the presence of the protein to investigate the affinity. 2D-[¹⁵N,¹H]-sofast HSQC experiments were acquired with 50 μM protein and 250 μM of compound, using 64 scans with 2048 and 128 complex data points in the ¹H and ¹³C dimensions, respectively, at 298 K.

### Denaturation Thermal Shift Measurements

Thermal shift assays for Pin1 alone and in the presence of the inhibitors were obtained with a BioRad CFX Connect Real-Time PCR Detection System. Each data point was collected in quadruplicate. Each sample was prepared using buffer 50 mM Phosphate pH 7.5, 150 mM NaCl, 1% DMSO. Protein: ligand ratio used was 1:2 (20 μM protein-40 μM compound), and SYPRO Orange was used. Incubation of the Pin1 protein with the compounds was performed at 10° C. for 10 min. Samples were then heated from 10 to 95° C. with heating increments of 0.05° C., over 30 min. Fluorescence intensity was measured within the excitation/emission ranges 470-505/540-700 nm.

### Delfia (Dissociation-Enhanced Lanthanide Fluorescent Immunoassay)

Each well of 96-well streptavidin-coated plates (PerkinElmer) was incubated with 100 μL of 200 nM biotinylated D-peptide for 2 h and washed 3 times with a wash solution (PerkinElmer). Subsequently, a mixture containing Pin1, and a serial dilution of the test compounds were pre-incubated for 6 h. Eu-N1-labeled anti-6x-His antibody (PerkinElmer, 1:2000) was then added to this mixture and further incubated in the biotinylated D-peptide-bound plates for another 2 h. At the end of the incubation, plates were washed 3 times and 200 μL of enhancement solution (PerkinElmer) was added to each well. After a 10-minute incubation, fluorescence measurements were taken with the Victor X5 microplate reader with the excitation and emission wavelengths of 340 and 615, respectively. The final Pin1 protein concentration used was 8 nM and all the incubations were performed at room temperature. Fluorescence readings were normalized to those of 1% DMSO-treated wells and reported as % inhibition. Prism 10 (GraphPad) was used to calculate IC₅₀ values.

### Cell Culture and Nuclear Labeling

BxPC3, MIA PaCa-2, PANC-1, MDA-MB-231, and PC3 were obtained from the American Type Culture Collection (ATCC) and A549 NucLight Red cells were purchased from Essen Bioscience. BxPC3 and PC3 cells were cultured in RPMI (Corning) while PANC-1 cells were cultured in DMEM (Corning). MIA PaCa-2 cells were also cultured in DMEM, but with the addition of 2.5% horse serum. A549 NucLight cells were cultured in a Ham's F-12 nutriend mixture with GlutaMAX-1 (Gibco). All of the culture media were supplemented with 10% FBS. MDA-MB-231 cells were nuclear-labeled red (MDA-MB-231 NucLight Red) with the IncuCyte NucLight lentivirus reagent (Essen Bioscience) according to the manufacturer's protocol and were cultured in DMEM supplemented with 10% FBS and 1 μg/mL puromycin. Cells were all maintained at 37° C. in a humidified incubator with 5% CO₂.

### Immunoblotting

At the end of the treatments, cells were lysed with a lysis buffer containing 20 mM Tris, pH 7.4, 120 mM NaCl, 1% Triton X-100, 0.5% sodium deoxycholate, 0.1% SDS, 1% IGEPAL, 5 mM EDTA, protease inhibitor cocktail and PhosSTOP (Sigma-Aldrich). Lysates were then centrifuged at 16,000×g for 20 min at 4° C. and supernatants were collected. Protein concentrations were determined using the Pierce BCA Protein Assay Kit (ThermoFisher Scientific) according to the manufacturer's instruction. Samples were then loaded onto precast Bis-Tris gels and subsequently transferred onto PVDF membranes. Blots were blocked with 5% nonfat milk for 1 h and incubated with anti-Pin1 (Cell Signaling Technology, #3722) or anti-β-actin (Santa Cruz Biotechnology, #sc-69879) antibodies at 4° C. overnight. Next day, blots were incubated with appropriate HRP-conjugated secondary antibodies for 1 h at room temperature and Clarity Western ECL solution (BIO-RAD) was added to the blots prior to being captured on a ChemiDoc imaging system (BIO-RAD).

Crystallizing and Soaking Apo R14A Pin1 with 158F10 and with 158D9.

The purified apo and compound-modified proteins were buffer exchanged into 20 mM HEPES-NaOH at pH 7.5, 100 mM NaCl, and 1 mM DTT by gel filtration, concentrated to 10-20 mg/mL, aliquoted, flash frozen in liquid nitrogen, and stored at −80° C. Apo R14A Pin1 was crystalized by vapor diffusion at 4° C. using a sitting drop format (Cryschem M plate, Hampton Research). A mixture of 1 μL of 10-20 mg/mL R14A Pin1 and 1 μL of reservoir solution was incubated under Argon over 500 μL of reservoir solution. The reservoir solution contained 100 mM HEPES-NaOH or 100 mM BisTris-HCl at pH 7.1-7.2, 2.47 M ammonium sulfate, 1% [v/v] PEG400 and 1 mM TCEP. Crystals appeared overnight and grew to a size of about 100 μm×50 μm×50 μm within 2-3 days. Crystals were soaked overnight to 24 hours with ˜ 4 μM of compound 158F10 or ˜0.14 μM of compound 158D9 in an artificial mother liquor consisting of 51% [v/v] reservoir solution and 49% [v/v] of 20 mM HEPES-NaOH at pH 7.5, 100 mM NaCl, and 1 mM DTT. The soaked crystals were stabilized in 2.8 M LiSO₄ before being flash-frozen in liquid nitrogen.

### Crystallizing 164A10-Modified Pin1 R14a.

164A10-modified R14A Pin1 was crystalized by vapor diffusion at 4° C. using a sitting drop format (Cryschem M plate, Hampton Research). A mixture of 1 μL of 15 mg/mL of 164A10-modified R14A hPin1 and 1 L of reservoir solution was incubated over 500 μL of reservoir solution. The reservoir solution contained 100 mM HEPES-NaOH at pH 7.4, 2.30 M ammonium sulfate, 1% [v/v] PEG400 and 1 mM DTT. Crystals that grew within a month to a size of about 200 μm×80 μm×80 μm were stabilized in 2.8 M LiSO₄ and flash-frozen in liquid nitrogen.

### Crystallizing 158F10-Modified R14a.

158F10-modified R14A Pin1 crystals were grown by vapor diffusion at 4° C. The initial crystals grew from a mixture of 1 μL of 10 mg/mL of 158A10-modified R14A hPin1 and 1 μL of reservoir solution using a hanging drop format (EasyXtal 15-well tool, Qiagen). The reservoir solution contained 100 mM HEPES-NaOH at pH 6.9, 2.30 M ammonium sulfate, 1% [v/v] PEG400 and 1 mM DTT. These initial crystals were used to streak seed a mixture of 1 μL of 20 mg/mL of 158F10-modified R14A hPin1 and 1 μL of reservoir solution was incubated over 500 μL reservoir solution that was incubated for 30 minutes. Crystals appeared within one to two days and reached full size (250 μm×50 μm×50 μm) within a few weeks. The reservoir solution contained 100 mM HEPES-NaOH at pH 6.9, 2.20 M ammonium sulfate, 1% [v/v] PEG400 and 1 mM DTT. Full-sized crystals were stabilized in 2.8 M LiSO₄ before being flash-frozen in liquid nitrogen.

### X-Ray Data Collection and Structure Determination.

Data were collected remotely at beamline 5.0.3 of the Advanced Light Source (ALS) at 0.9748 Å wavelength and 100 K. Diffraction data were processed with XDS (Kabsch W (2010) Xds. Acta Crystallogr D Biol Crystallogr 66(Pt 2):125-132) and with the CCP4 programs Aimless and Pointless (Winn M D, et al. (2011) Acta Crystallogr D Biol Crystallogr 67(Pt 4):235-242; Evans P (2006) Acta Crystallogr D Biol Crystallogr 62(Pt 1):72-82; and Evans P R & Murshudov G N (2013) Acta Crystallogr D Biol Crystallogr 69(Pt 7):1204-1214). The structures were solved by molecular replacement with the CCP4 program Phaser (Agirre J, et al. (2023) Acta Crystallogr D Struct Biol 79(Pt 6):449-461; and McCoy A J, et al. (2007) J Appl Crystallogr 40(Pt 4):658-674) using the structure R14A hPin1 as search model (PDB-ID 6033) (Pinch B J, et al. (2020) Nat Chem Biol 16(9):979-987). The structure of each inhibitor modified protein was generated in an iterative process of rebuilding structure in COOT and refining each rebuild structure with phenix.refine from the PHENIX suite (Emsley P, Lohkamp B, Scott W G, & Cowtan K (2010) Acta Crystallogr D Biol Crystallogr 66(Pt 4):486-501; and Liebschner D, et al. (2019) Acta Crystallogr D Struct Biol 75(Pt 10):861-877) Stereochemical restraints for the different inhibitors were generated using phenix.elbow also from the PHENIX suit. Figures were prepared using ChimeraX v.1.7 (Meng E C, et al. (2023) Protein Sci 32(11):e4792). Feature-enhanced maps (Afonine P V, et al. (2015) Acta Crystallogr D Biol Crystallogr 71(Pt 3):646-666) were used for later stages of model building. phenix.refine using 2mFODFc maps and generated Feature Enhanced Maps using model phases. Statistics of data collection and refinement are summarized in Table S3.

TABLE S3
X-ray crystallography data collection and refinement statistics.

| Compound (PDB ID) | | | |
|---|---|---|---|
| 164A10 | 158F10 | 158D9 | 158F10 |
| (8VJG) | (8VJE) | (8VJF) | (8VJD) |

PDB ID

| Data collection | | | | | | | | |
|---|---|---|---|---|---|---|---|---|
| Space group | P 3₁ 2 1 | | P 3₁ 2 1 | | P 3₁ 2 1 | | P 2₁ 2₁ 2₁ | |
| Molecules in ASU | 1 | | 1 | | 1 | | 2 | |
| Cell dimensions | | | | | | | | |
| a, b, c (Å) | 68.83 68.83 | | 69.11 69.11 | | 68.87 68.87 | | 60.00 62.03 | |
| | 79.25 | | 79.03 | | 78.74 | | 93.41 | |
| α, β, γ (°) | 90.00 90.00 | | 90.00 90.00 | | 90.00 90.00 | | 90.00 90.00 | |
| | 120.00 | | 120.00 | | 120.00 | | 90.00 | |
| Resolution (Å)* | 47.64-1.58 | | 47.71-1.70 | | 47.54-1.70 | | 46.71-1.57 | |
| | (1.61-1.58) | | (1.73-1.70) | | (1.73-1.70) | | (1.60-1.57) | |
| R_{merge} (%)* | 3.2 | (81.9) | 4.8 | (102.6) | 8.2 | (105.6) | 6.2 | (62.9) |
| I/σI* | 36.8 | (2.3) | 30.7 | (2.2) | 25.9 | (2.4) | 17.6 | (2.3) |
| Completeness (%)* | 99.9 | (99.7) | 100.0 | (100.0) | 100.0 | (100.0) | 99.9 | (99.5) |
| Redundancy* | 9.6 | (8.6) | 9.7 | (9.8) | 9.5 | (9.7) | 6.4 | (5.2) |
| Refinement | | | | | | | | |
| No. reflections | 30,191 | | 24,551 | | 24,276 | | 49,405 | |
| R_{work} | 0.2010 | | 0.1985 | | 0.2143 | | 0.1986 | |
| R_{free} | 0.2308 | | 0.2266 | | 0.2548 | | 0.2151 | |
| No. atoms | | | | | | | | |
| Protein | 1,164 | | 1,164 | | 1,164 | | 2,328 | |
| Ligand | 42/22/8 | | 39/22/8 | | 38/22/8 | | 78/44/0 | |
| (Inhibitor/PEG/DTT) | | | | | | | | |
| Water | 158 | | 157 | | 156 | | 274 | |
| RMSD | | | | | | | | |
| Bond length (Å) | 0.007 | | 0.007 | | 0.007 | | 0.006 | |
| Bond angles (°) | 1.0 | | 1.0 | | 0.9 | | 0.9 | |
| Ramachandran | | | | | | | | |

| | | | | |
|---|---|---|---|---|
| Favored (%) | 100.00 | 98.58 | 100.00 | 99.29 |
| Allowed (%) | 0.00 | 1.42 | 0.00 | 0.71 |
| Outliers (%) | 0.00 | 0.00 | 0.00 | 0.00 |

*Values in parentheses are for highest-resolution shell.
Abbreviations:
ACN: acetonitrile;
DELFIA: Dissociation-Enhanced Lanthanide Fluorescent Immunoassay;
DMF: Dimethylformamide;
HATU: 1-[Bis(dimethylamino)methylene]-1H-1,2,3-triazolo[4,5-b]pyridinium 3-oxid hexafluorophosphate;
DIPEA: N,N-diisopropylethylamine;
DBU: 1,8-Diazabicyclo[5.4.0]undec-7-ene;
DTT: Dithiothreitol;
OXYMA: Ethyl cyanoglyoxylate-2-oxime;
THF: Tetrahydrofuran.

### Example 2

Additional data for representative compounds of the invention was generated using methods similar to those described in Example 1 and is provided in the following table.

TABLE 5

| Name | Structure | DELFIA IC₅₀ (nM) | ΔTₘ (° C.) |
|---|---|---|---|
| 158G6 | 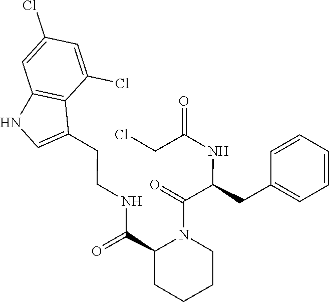 [SMILES: O=C(CCl)N[C@@H](Cc1ccccc1)C(=O)N1CCCC[C@H]1C(=O)NCCc1c[nH]c2cc(Cl)cc(Cl)c12] | 327 ± 18 | +1.04 ± 0.08 |
| 158G12 | 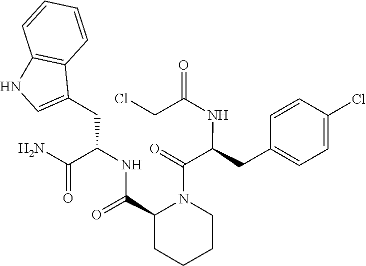 [SMILES: NC(=O)[C@H](Cc1c[nH]c2ccccc12)NC(=O)[C@@H]1CCCCN1C(=O)[C@H](Cc1ccc(Cl)cc1)NC(=O)CCl] | 527 ± 38 | −12.19 ± 0.07 |
| 164A1 | 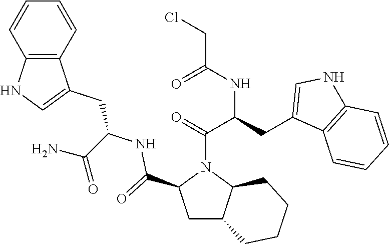 [SMILES: NC(=O)[C@H](Cc1c[nH]c2ccccc12)NC(=O)[C@@H]1C[C@H]2CCCC[C@@H]2N1C(=O)[C@H](Cc1c[nH]c2ccccc12)NC(=O)CCl] | 1826 ± 394 | −23.86 ± 0.35 |
| 164A2 | 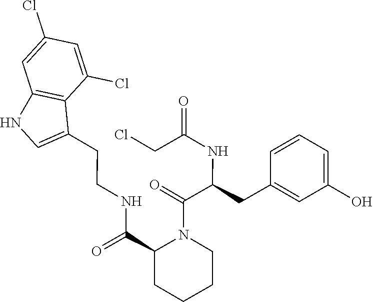 [SMILES: O=C(CCl)N[C@@H](Cc1cccc(O)c1)C(=O)N1CCCC[C@H]1C(=O)NCCc1c[nH]c2cc(Cl)cc(Cl)c12] | 289 ± 23 | −20.10 ± 0.05 |
| 164A3 | 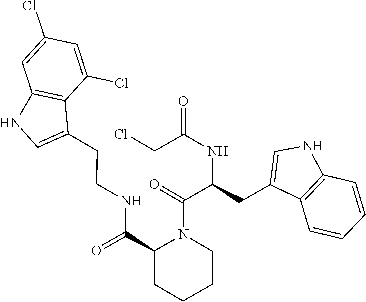 [SMILES: O=C(CCl)N[C@@H](Cc1c[nH]c2ccccc12)C(=O)N1CCCC[C@H]1C(=O)NCCc1c[nH]c2cc(Cl)cc(Cl)c12] | 1175 ± 45 | −13.68 ± 0.20 |
| 164A5 | 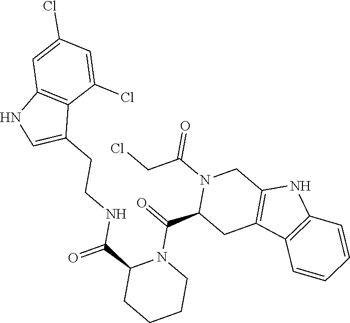 [SMILES: O=C(NCCc1c[nH]c2cc(Cl)cc(Cl)c12)[C@@H]1CCCCN1C(=O)[C@@H]1Cc2c([nH]c3ccccc23)CN1C(=O)CCl] | 433 ± 65 | +0.66 ± 0.28 |

Denaturation thermal shift data of selected agents. Table listing chemical structures, IC₅₀ values, and ΔTₘ values of agents 158G6, 158G12, 164A1, 164A2, 164A3, and 164A5 (also see Figure 12).

### Example 3. Preparation of Bifunctional Conjugates

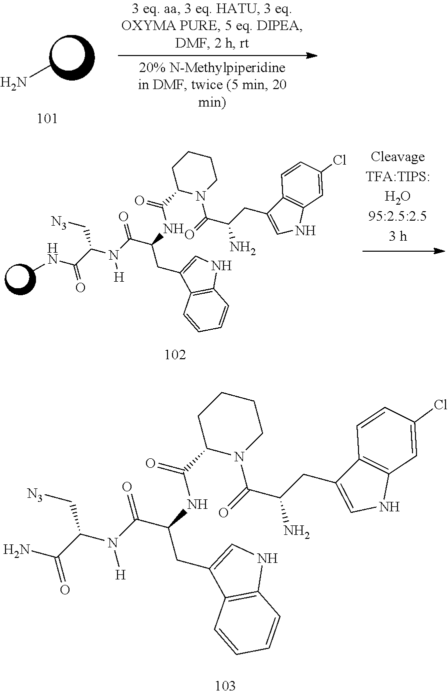

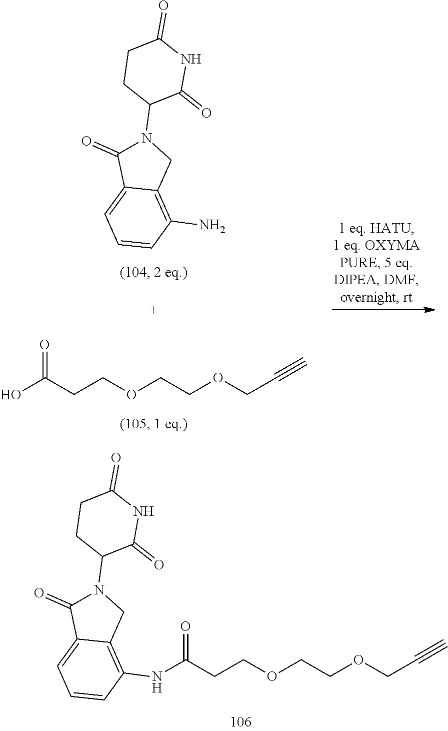 [SMILES: Nc1cccc2c1CN(C1CCC(=O)NC1=O)C2=O ; C#CCOCCOCCC(=O)O ; C#CCOCCOCCC(=O)Nc1cccc2c1CN(C1CCC(=O)NC1=O)C2=O]

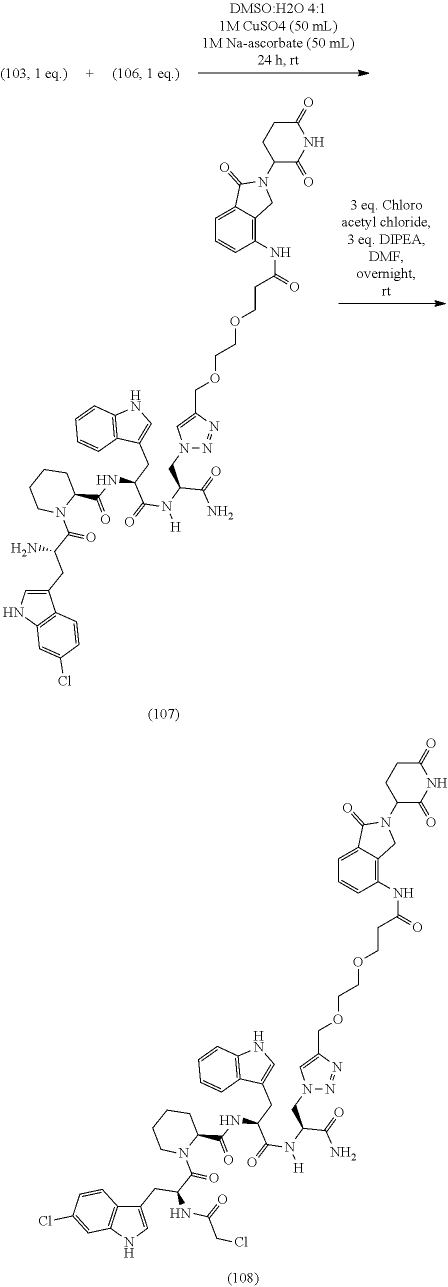 [SMILES: NC(=O)[C@H](Cn1cc(COCCOCCC(=O)Nc2cccc3c2CN(C2CCC(=O)NC2=O)C3=O)nn1)NC(=O)[C@H](Cc1c[nH]c2ccccc12)NC(=O)[C@@H]1CCCCN1C(=O)[C@@H](N)Cc1c[nH]c2cc(Cl)ccc12 ; NC(=O)[C@H](Cn1cc(COCCOCCC(=O)Nc2cccc3c2CN(C2CCC(=O)NC2=O)C3=O)nn1)NC(=O)[C@H](Cc1c[nH]c2ccccc12)NC(=O)[C@@H]1CCCCN1C(=O)[C@H](Cc1c[nH]c2cc(Cl)ccc12)NC(=O)CCl]

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A compound or conjugate having structure of Formula (I) 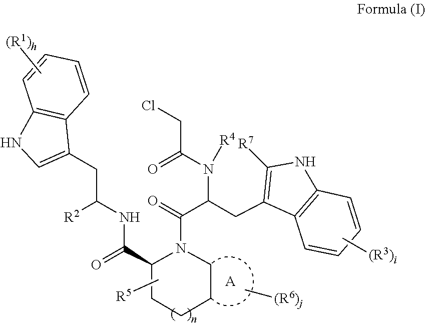
or a salt thereof, wherein
each R¹ is independently halo, hydroxy, (C₁-C₆)alkyl, (C₁-C₆)alkoxy, —CN, —NO₂, —SO₂F, —OSO₂F, or (C₁-C₆)alkyl-(C₁-C₆)alkoxy;
R² is H, (C₁-C₆)alkyl, aryl, heteroaryl, heterocycle, —C(═O)-L-D, —C(═O)NH-L-D, —C(═O)NH₂, or —C(═O)NH—Rₐ, wherein the (C₁-C₆)alkyl, aryl, heteroaryl, or heterocycle is optionally substituted with one or more substituents selected from the group consisting of halo, hydroxy, (C₁-C₆)alkyl, (C₁-C₆)alkoxy, —CN, —NO₂, —SO₂F, and —OSO₂F;
each R³ is independently halo, hydroxy, (C₁-C₆)alkyl, (C₁-C₆)alkoxy, —CN, —NO₂, —SO₂F, —OSO₂F, or (C₁-C₆)alkyl-(C₁-C₆)alkoxy;
h, i, and j are each independently 0, 1, 2, 3, or 4;
R⁴ is H, (C₁-C₆)alkyl, (C₂-C₆)alkenyl, or (C₂-C₆)alkynyl and R⁷ is H; or R⁴ and R⁷ taken together are —CH₂—;
R⁵ is absent, halo, (C₁-C₆)alkyl, (C₁-C₆)alkoxy, —CN, —NO₂, —SO₂F, —OSO₂F, (C₁-C₆)alkyl-(C₁-C₆)alkoxy, aryl, heteroaryl, or heterocycle;
n is 0 or 1;
ring A is absent or a (C₃-C₆) carbocycle ring;
each R⁶ is independently halo, hydroxy, (C₁-C₆)alkyl, (C₁-C₆)alkoxy, —CN, —NO₂, —SO₂F, or —OSO₂F;
Rₐ is (C₁-C₆)alkyl, aryl, heteroaryl, or heterocycle, wherein the (C₁-C₆)alkyl, aryl, heteroaryl, or heterocycle is optionally substituted with one or more substituents selected from the group consisting of halo, hydroxy, (C₁-C₆)alkyl, (C₁-C₆)alkoxy, —CN, —NO₂, —SO₂F, and —OSO₂F;
L is absent or is a linking group; and
D is the residue of an E3 ligase targeting drug moiety, the residue of another molecule targeting PIN1, or the residue of a ligand targeting a different protein target to induce its PIN1 driven degradation.

2. The compound or conjugate of claim 1, having structure of Formula (Ia) or (Ib) 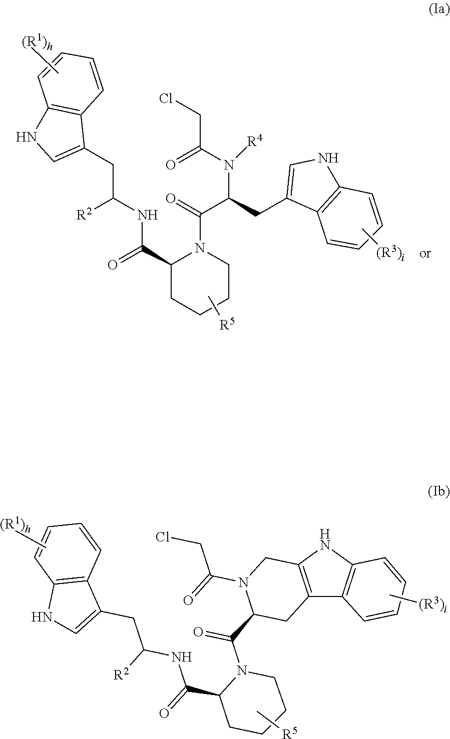
or salt thereof.

3. The compound, conjugate, or salt of claim 1, wherein each R¹ is independently —Cl, —F, —CH₃, —C₂H₅, —CH(CH₃)₂, —CH₂CH₂CH₃, —OCH₃, —OC₂H₅, —CN, —NO₂, —SO₂F, —OSO₂F, or —CH₂—O—CH₃.

4. The compound or salt of claim 1, wherein R² is-C(═O)NH₂.

5. The conjugate or salt of claim 1, wherein, R² is-C(—O)-L-D or —C(═O)NH-L-D.

6. The compound, conjugate, or salt of claim 1, wherein each R³ is independently, —Cl, —F, —CH₃, —C₂H₅, —CH(CH₃)₂, —CH₂CH₂CH₃, —OCH₃, —OC₂H₅, —CN, —NO₂, —SO₂F, —OSO₂F, or —CH₂—O—CH₃.

7. The compound, conjugate, or salt of claim 1, wherein n is 0, and ring A is a (C₃-C₆) carbocycle ring.

8. The compound, conjugate, or salt of claim 1, wherein each R⁶ is independently halo, or (C₁-C₆)alkyl.

9. The conjugate or salt of claim 1, which is a conjugate of Formula (Ic): 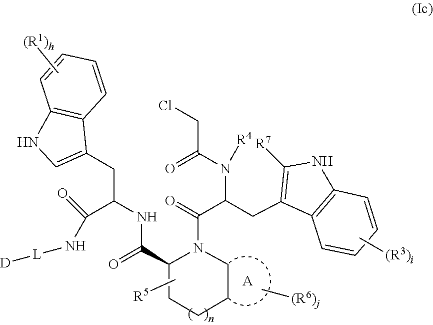
or a salt thereof, wherein:
L is absent or a linker;
D is the residue of an E3 ligase targeting drug moiety, the residue of another molecule targeting PIN1, or the residue of a ligand targeting a different protein target to induce its PIN1 driven degradation;
each R¹ is independently halo, hydroxy, (C₁-C₆)alkyl, (C₁-C₆)alkoxy, —CN, —NO₂, —SO₂F, —OSO₂F, or (C₁-C₆)alkyl-(C₁-C₆)alkoxy;
each R³ is independently halo, hydroxy, (C₁-C₆)alkyl, (C₁-C₆)alkoxy, —CN, —NO₂, —SO₂F, —OSO₂F, or (C₁-C₆)alkyl-(C₁-C₆)alkoxy;
h, i, and j are each independently 0, 1, 2, 3, or 4;
R⁴ is H, (C₁-C₆)alkyl, (C₂-C₆)alkenyl, or (C₂-C₆)alkynyl, and R⁷ is H; or R⁴ and R⁷ taken together are —CH₂— (i.e., R⁴ and R⁷ along with the intervening atoms form a 6-membered ring);
R⁵ is absent, halo, (C₁-C₆)alkyl, (C₁-C₆)alkoxy, —CN, —NO₂, —SO₂F, —OSO₂F, (C₁-C₆)alkyl-(C₁-C₆)alkoxy, aryl, heteroaryl, or heterocycle;
n is 0 or 1;
ring A is absent or a (C₃-C₆) carbocycle ring;
each R⁶ is independently halo, hydroxy, (C₁-C₆)alkyl, (C₁-C₆)alkoxy, —CN, —NO₂, —SO₂F, or —OSO₂F; and
Rₐ is (C₁-C₆)alkyl, aryl, heteroaryl, or heterocycle, wherein the (C₁-C₆)alkyl, aryl, heteroaryl, or heterocycle is optionally substituted with one or more substituents selected from the group consisting of halo, hydroxy, (C₁-C₆)alkyl, (C₁-C₆)alkoxy, —CN, —NO₂, —SO₂F, and —OSO₂F.

10. The conjugate or salt of claim 9, wherein L comprises a branched or unbranched, saturated or unsaturated, hydrocarbon chain, having from about 1 to 25 carbon atoms, wherein one or more of the carbon atoms is optionally replaced independently by —O—, —S, —N(R^{a})₂, —N(R^{a})—, 3-7 membered heterocycle, 5-6-membered heteroaryl or carbocycle and wherein each chain, 3-7 membered heterocycle, 5-6-membered heteroaryl or carbocycle is optionally and independently substituted with one or more (e.g. 1, 2, 3, 4, 5 or more) substituents selected from (C₁-C₆)alkyl, (C₁-C₆)alkoxy, (C₃-C₆) cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, —N(R^{a})₂, hydroxy, oxo (═O), carboxy, aryl, aryloxy, heteroaryl, and heteroaryloxy, wherein each R^{a} is independently H or (C₁-C₆)alkyl.

11. The conjugate or salt of claim 9, wherein L comprises a branched or unbranched, saturated or unsaturated, hydrocarbon chain, having from about 1 to 25 carbon atoms, wherein one or more of the carbon atoms is optionally replaced independently by a group selected from the group consisting of —O—, —C(═O)—, —S, —N(R^{a})₂, and —N(R^{a})—, wherein each R^{a} is independently h or (C₁-C₆)alkyl.

12. The conjugate or salt of claim 9, wherein L is selected from the group consisting of:

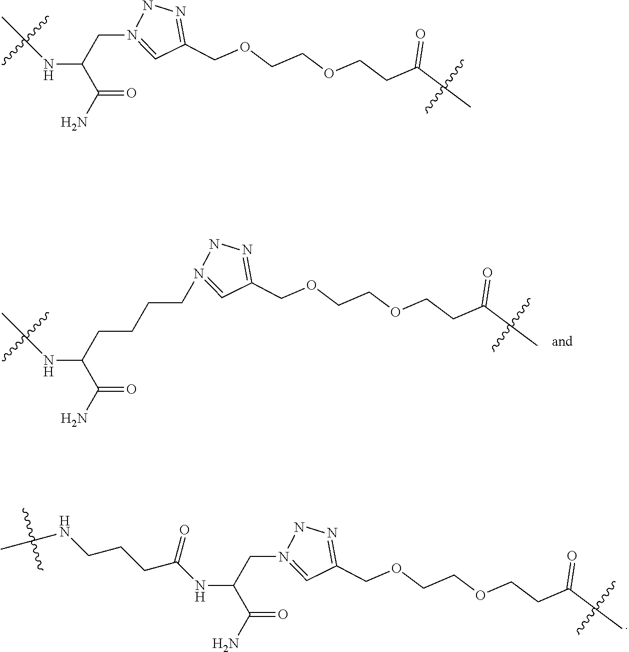

13. The compound of claim 1, which is selected from the group consisting of: 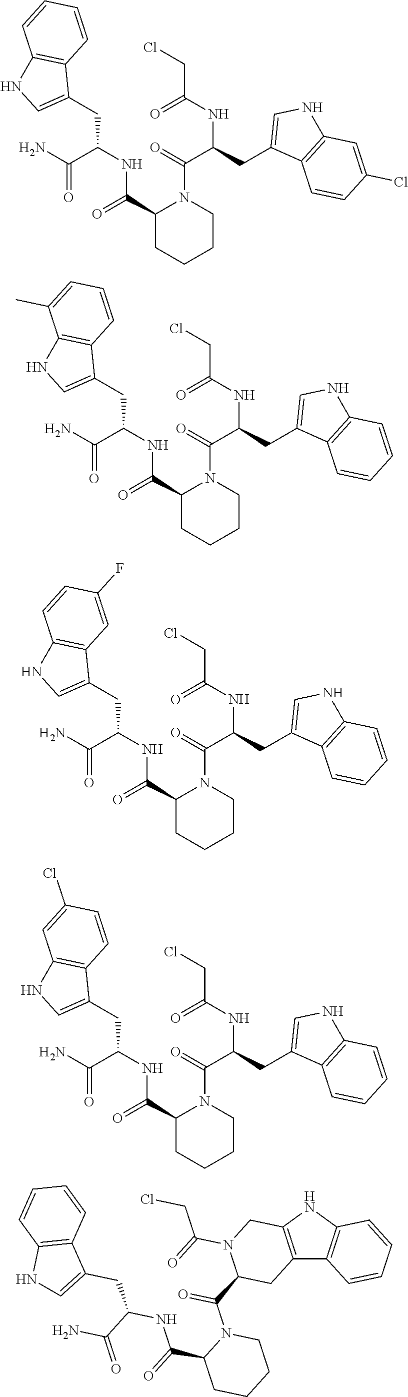 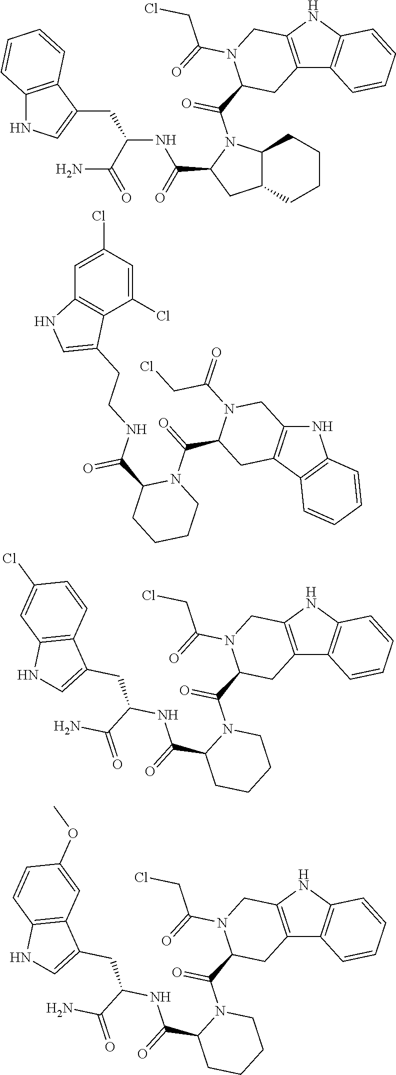 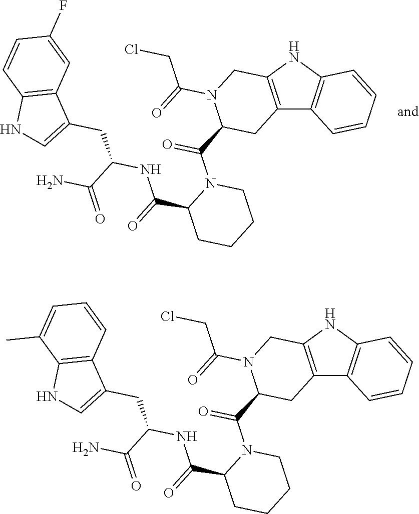 [SMILES: NC(=O)[C@H](Cc1c[nH]c2ccc(F)cc12)NC(=O)[C@@H]1CCCCN1C(=O)[C@@H]1Cc2c([nH]c3ccccc23)CN1C(=O)CCl ; Cc1cccc2c(C[C@H](NC(=O)[C@@H]3CCCCN3C(=O)[C@@H]3Cc4c([nH]c5ccccc45)CN3C(=O)CCl)C(N)=O)c[nH]c12]
or salt thereof.

14. The compound of claim 1, which is selected from the group consisting of: 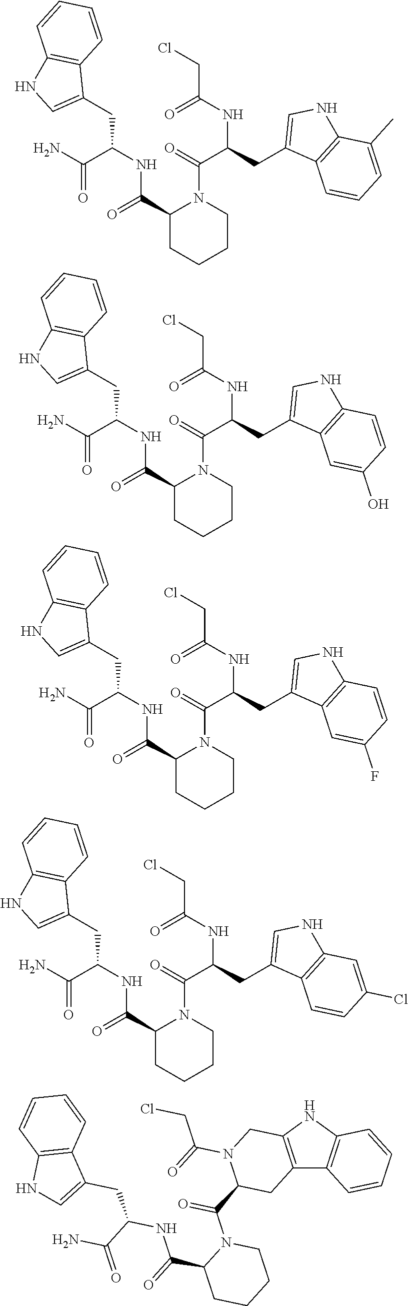 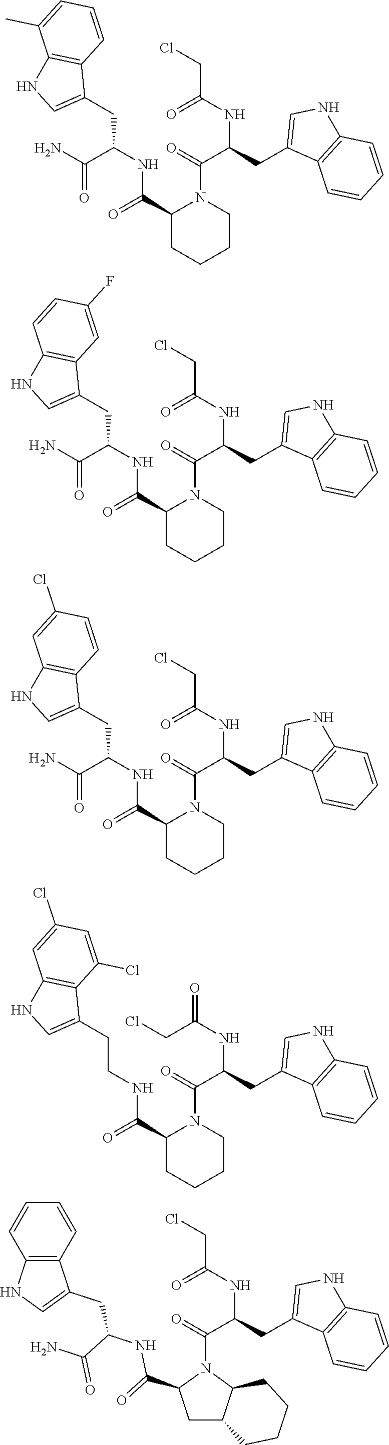 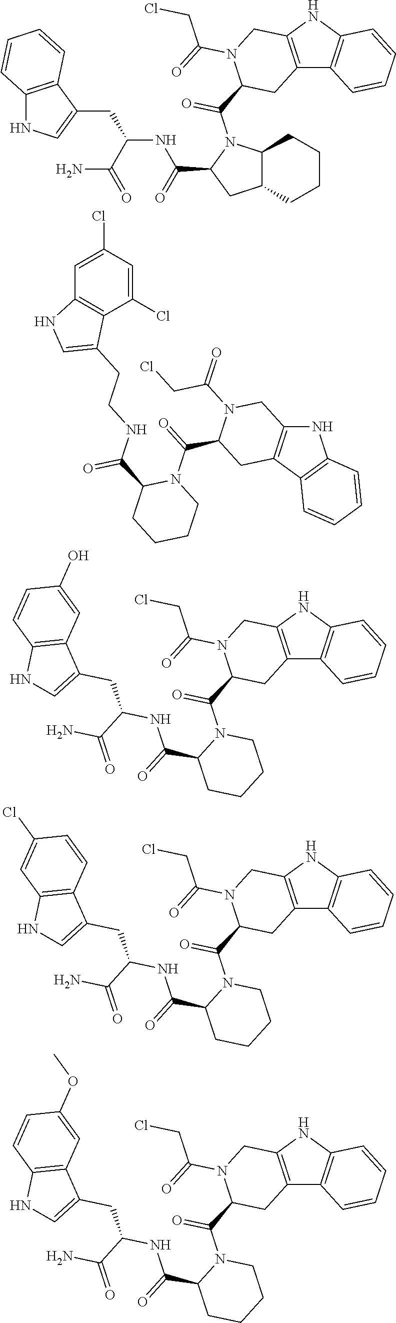 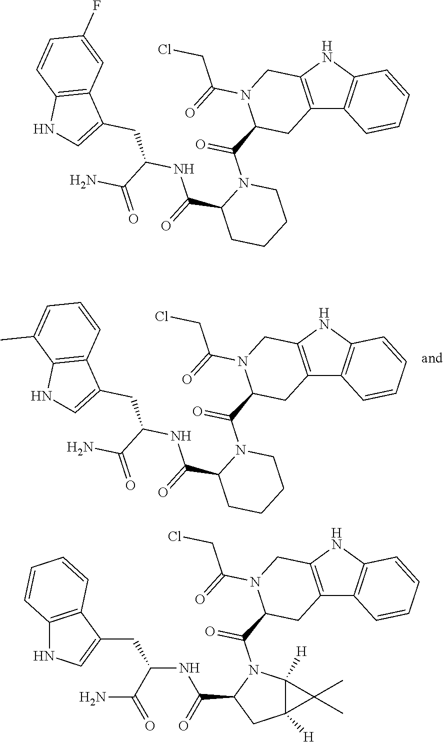
or a salt thereof.

15. The compound of claim 1, which is: 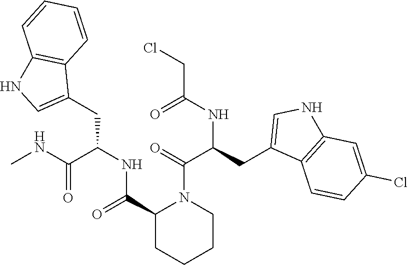 [SMILES: CNC(=O)[C@H](Cc1c[nH]c2ccccc12)NC(=O)[C@@H]1CCCCN1C(=O)[C@H](Cc1c[nH]c2cc(Cl)ccc12)NC(=O)CCl]
or a salt thereof.

16. The compound of claim 1, which is selected from the group consisting of: 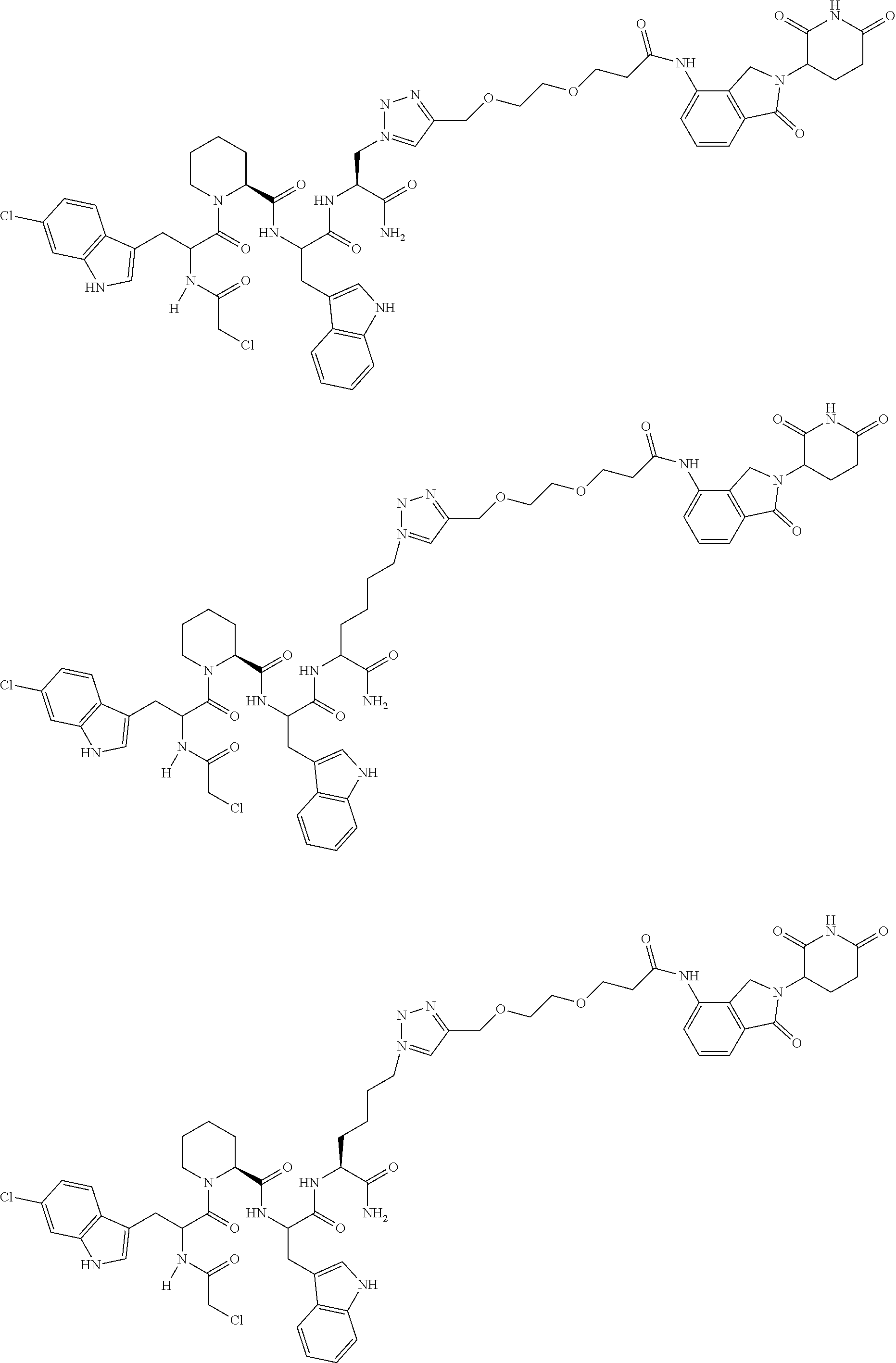 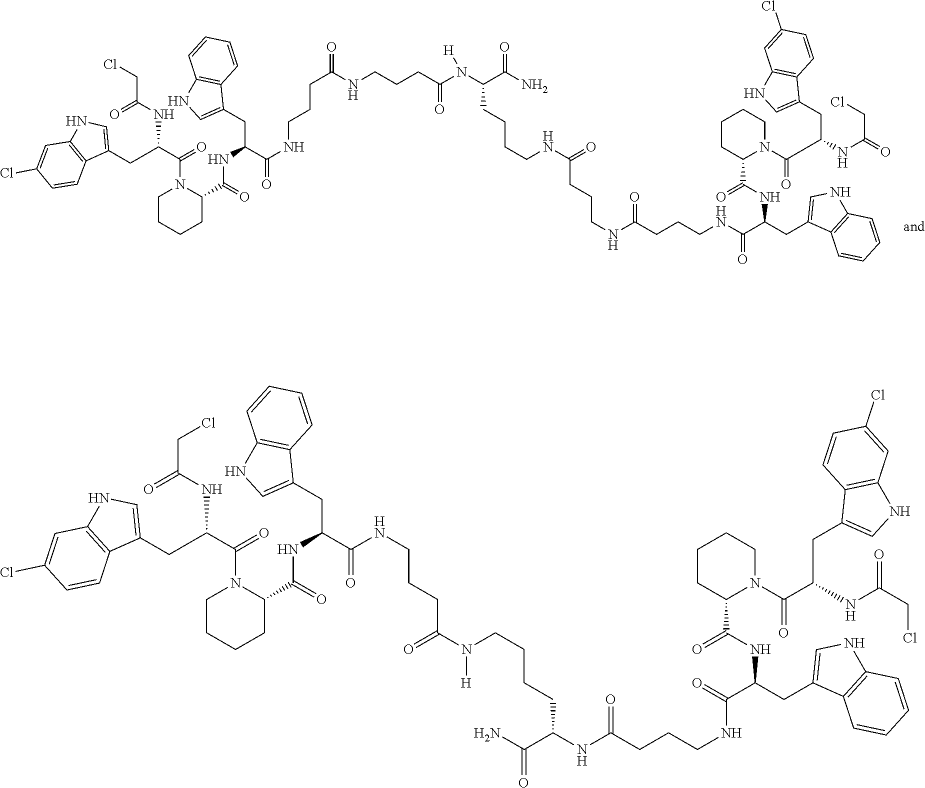 [SMILES: NC(=O)[C@H](CCCCNC(=O)CCCNC(=O)CCCNC(=O)[C@H](Cc1c[nH]c2ccccc12)NC(=O)[C@@H]1CCCCN1C(=O)[C@H](Cc1c[nH]c2cc(Cl)ccc12)NC(=O)CCl)NC(=O)CCCNC(=O)CCCNC(=O)[C@H](Cc1c[nH]c2ccccc12)NC(=O)[C@@H]1CCCCN1C(=O)[C@H](Cc1c[nH]c2cc(Cl)ccc12)NC(=O)CCl ; NC(=O)[C@H](CCCCNC(=O)CCCNC(=O)[C@H](Cc1c[nH]c2ccccc12)NC(=O)[C@@H]1CCCCN1C(=O)[C@H](Cc1c[nH]c2cc(Cl)ccc12)NC(=O)CCl)NC(=O)CCCNC(=O)[C@H](Cc1c[nH]c2ccccc12)NC(=O)[C@@H]1CCCCN1C(=O)[C@H](Cc1c[nH]c2cc(Cl)ccc12)NC(=O)CCl]
or a salt thereof.

17. A pharmaceutical composition comprising a compound, conjugate, or salt as described in claim 1 and a pharmaceutically acceptable carrier.

18. A method of inhibiting and/or degrading PIN1 in vitro or in vivo, comprising contacting PIN1 with a compound, conjugate, or salt as described in claim 1.

19. A method of treating a PIN1 associated disease in a mammal in need thereof, comprising administering a therapeutically effective amount of a compound, conjugate, or salt as described in claim 1, to the mammal.

20. A method of developing degrader compound for a target protein, comprising
contacting a test compound with the target protein in vitro,
determining the binding affinity of the test compound for the target protein,
determining the thermal stability of the target protein in the absence and presence of the test compound,
identifying the test compound as a degrader compound wherein the test compound is determined to be capable of binding the target protein and is determined to be capable of reducing the thermal stability of the target protein.
